(19) United States
(12) Patent Application Publication
(10) Pub. No.: US 2021/0348178 A1
(43) Pub. Date: Nov. 11, 2021

(54) COMPOSITIONS AND METHODS FOR MODIFYING MATURITY IN RICE PLANTS

(71) Applicant: PIONEER HI-BRED INTERNATIONAL, INC., JOHNSTON, IA (US)

(72) Inventors: AMITABH MOHANTY, JOHNSTON, IA (US); BHOJARAJA RUDRAPPA, HYDERABAD (IN); VENKATA S TAVVA, HYDERABAD (IN)

(73) Assignee: PIONEER HI-BRED INTERNATIONAL, INC., JOHNSTON, IA (US)

(21) Appl. No.: 17/269,342

(22) PCT Filed: Aug. 15, 2019
(86) PCT No.: PCT/US2019/046608

§ 371 Date: Feb. 18, 2021

Related U.S. Application Data
(60) Provisional application No. 62/720,522, filed on Aug. 21, 2018.

(51) Int. Cl. C12N 15/82

(52) U.S. Cl. CPC C12N 15/827

(57) ABSTRACT

Methods and compositions to modulate maturity of a rice plant are disclosed. The disclosure further discloses compositions, polynucleotide constructs, transformed host cells, plants and seeds exhibiting altered stature characteristics or produce plants that exhibit altered maturity parameters.

### FIELD

This disclosure relates to compositions and methods of modifying maturity in rice plants.

### REFERENCE TO SEQUENCE LISTING SUBMITTED ELECTRONICALLY

The official copy of the sequence listing is submitted electronically via EFS-Web as an ASCII formatted sequence listing with a file named “7438_ST25.txt” created on Aug. 17, 2018 and having a size of 123.4 kilobytes and is filed concurrently with the specification. The sequence listing contained in this ASCII formatted document is part of the specification and is herein incorporated by reference in its entirety.

### BACKGROUND

Recent advances in plant genetic engineering have opened new doors to engineer plants to have improved characteristics or traits, such as maturity, stature, height and other architecture. Early or late maturity, depending on the need, is a desirable trait in crop breeding for a variety of crops of commercial interest. Maturity adaptations increase harvest index, favorably partition carbon and nutrients between grain and non-grain biomass, enhance fertilizer use, water use efficiency and play a role in increasing planting density.

### SUMMARY

Provided herein is a method of modifying maturity, the method comprising introducing one or more nucleotide modifications through a targeted DNA break at a genomic locus of a plant, wherein the genomic locus comprises a polynucleotide involved in flowering time regulation (FTR) encoding a response regulator receiver domain containing protein, a CCT motif containing protein, BHLH transcription factor, TCP family transcription factor, NAC domain-containing protein, tubulin/FtsZ domain containing protein, hsp20/alpha crystallin protein, core histone H2A/H2B/H3/H4 putative protein, AAA-type ATPase family protein, universal stress protein domain containing protein, PHD finger family protein, or a methyl-binding domain protein, and wherein the plant maturity is modified compared to a control plant not comprising the one or more introduced genetic modifications. In certain embodiments, the FTR polynucleotide encodes a polypeptide comprising an amino acid sequence that is at least 90% identical to a sequence selected from the group consisting of SEQ ID NOs: 14-26. In certain embodiments, the targeted DNA modification targets more than one distinct genomic loci that is involved in FTR of the plant.

In certain embodiments, the targeted DNA modification is selected from the group consisting of insertion, deletion, single nucleotide polymorphism (SNP), and a polynucleotide modification, such that the expression of the FTR polypeptide is reduced. In certain embodiments, the targeted DNA modification results in one or more of the following: reduced expression of the FTR polynucleotide; reduced transcriptional activity of the protein encoded by the FTR polynucleotide; generation of one or more alternative spliced transcripts of the FTR polynucleotide; deletion of one or more DNA binding domains; frameshift mutation in one or more exons of the FTR polynucleotide; deletion of a substantial portion of the FTR polynucleotide or deletion of the full-length open reading frame of the FTR polynucleotide; repression of an enhancer motif present within a regulatory region encoding the FTR polynucleotide; modification of one or more nucleotides or deletion of a regulatory element operably linked to the expression of the FTR polynucleotide, wherein the regulatory element is present within a promoter, intron, 3′UTR, terminator or a combination thereof. In certain embodiments, the targeted DNA modification targets the genomic locus of the FTR polynucleotide such that the one or more nucleotide modifications are present within (a) the same coding region; (b) non-coding region; (c) regulatory sequence; (d) untranslated region, or (e) any combination of (a)-(d) of an endogenous polynucleotide encoding a polypeptide that is involved in maturity.

In certain embodiments, the targeted DNA modification is introduced by a RNA-guided endonuclease, a site-specific deaminase, or a site-specific endonuclease. In certain embodiments, the targeted DNA modification is through a genome modification technique selected from the group consisting of polynucleotide-guided endonuclease, CRISPR-Cas endonucleases, base editing deaminases, zinc finger nuclease, a transcription activator-like effector nuclease (TALEN), engineered site-specific meganucleases, or Argonaute. In certain embodiments, the targeted DNA modification is induced by using a guide RNA that corresponds to a target sequence comprising a polynucleotide that encodes a polypeptide comprising an amino acid sequence that is at least 90% identical to a sequence selected from the group consisting of SEQ ID NOS: 14-26.

In certain embodiments, the plant exhibits early maturity when the targeted DNA modification results in reduced expression or activity of the protein encoded by the FTR polynucleotide. In an embodiment the plant exhibits first flowering about 5 to 15 days earlier than a control plant.

In certain embodiments, the plant exhibits delayed maturity when the targeted DNA modification results in reduced expression or activity of the protein encoded by the FTR polynucleotide. In an embodiment, maturity of the plant is delayed by about 5% to about 50% compared to a control plant as measured by the number of days to first or 50% flowering or panicle emergence or panicle initiation.

In certain embodiments, the modification in maturity is obtained in the absence of a substantial reduction in grain yield measure per plant or as a population of plants per unit area. In certain embodiments, the stature of the plant is not significantly altered compared to the control rice plant as measured by a reduction in plant height. In certain embodiments, the targeted DNA modification of the plant does not substantially alter root architecture of the plant or does not significantly increase root lodging, compared to a control plant not comprising the modification(s).

In certain embodiments, the plant is a rice plant. In certain embodiments, the rice plant is a female in bred line. In certain embodiments, the rice plant is a hybrid. In certain embodiments, the rice plant is Oryza sativa var. indica.

Also provided herein is a rice plant exhibiting early maturity comprising a modified genomic locus involved in flowering time regulation (FTR), wherein the genomic locus comprises one or more introduced mutations compared to a control plant and wherein the FTR genomic locus encodes a polypeptide that is at least 90% identical to the amino acid sequence selected from the group consisting of SEQ ID NOS: 14-21. In certain embodiments, the rice plant exhibits early maturity in a range of about 5 to 15 days as measured by first flowering timing. In certain embodiments, the rice plant is a female in bred line. In certain embodiments, the rice plant is a hybrid. In certain embodiments, the rice plant is Oryza sativa var. indica. In certain embodiments, the modification in maturity is obtained in the absence of a substantial reduction in grain yield measure per plant or as a population of plants per unit area. In certain embodiments, the stature of the plant is not significantly altered compared to the control rice plant as measured by a reduction in plant height. In certain embodiments, the targeted DNA modification of the plant does not substantially alter root architecture of the plant or does not significantly increase root lodging, compared to a control plant not comprising the modification(s).

Provided herein is a rice plant exhibiting delayed maturity comprising a modified genomic locus involved in flowering time regulation (FTR), wherein the genomic locus comprises one or more introduced mutations compared to a control plant and wherein the FTR genomic locus encodes a polypeptide that is at least 90% identical to the amino acid sequence selected from the group consisting of SEQ ID NOS: 22-26. In certain embodiments, the maturity of the rice plant is delayed by about 5% to about 50% compared to the control rice plant as measured by the number of days to first or 50% flowering or panicle emergence or panicle initiation. In certain embodiments, the rice plant is a female in bred line. In certain embodiments, the rice plant is a hybrid. In certain embodiments, the rice plant is Oryza sativa var. indica. In certain embodiments, the modification in maturity is obtained in the absence of a substantial reduction in grain yield measure per plant or as a population of plants per unit area. In certain embodiments, the stature of the plant is not significantly altered compared to the control rice plant as measured by a reduction in plant height. In certain embodiments, the targeted DNA modification of the plant does not substantially alter root architecture of the plant or does not significantly increase root lodging, compared to a control plant not comprising the modification(s).

Also provided herein is a recombinant DNA construct comprising a polynucleotide sequence encoding an amino acid sequence that is at least 95% identical to an amino acid sequence selected from the group consisting of SEQ ID NOs: 14-26, operably linked to at least one heterologous nucleic acid sequence.

Additionally, provided herein is a plant cell comprising a recombinant DNA construct comprising a polynucleotide sequence encoding an amino acid sequence that is at least 95% identical to an amino acid sequence selected from the group consisting of SEQ ID NOs: 14-26, operably linked to at least one heterologous nucleic acid sequence.

Provided herein is a guide RNA sequence that targets a genomic locus of a plant cell, wherein the genomic locus comprises a polynucleotide that encodes a polypeptide comprising an amino acid sequence that is at least 90% identical to a sequence selected from the group consisting of SEQ ID NOS: 14-26. In certain embodiments, the guide RNA is present in a recombinant DNA construct.

Also provide are plant cells comprising a guide RNA sequence that targets a genomic loci of a plant cell, wherein the genomic loci comprises a polynucleotide that encodes a polypeptide comprising an amino acid sequence that is at least 90% identical to a sequence selected from the group consisting of SEQ ID NOS: 14-26, and plant cells comprising a recombinant DNA construct comprising a guide RNA sequence that targets a genomic loci of a plant cell, wherein the genomic loci comprises a polynucleotide that encodes a polypeptide comprising an amino acid sequence that is at least 90% identical to a sequence selected from the group consisting of SEQ ID NOS: 14-26.

Further provided herein is a plant, or seed produced therefrom, having stably incorporated into its genome a recombinant DNA construct comprising a guide RNA sequence that targets a genomic loci of the plant cell, wherein the genomic loci comprises a polynucleotide that encodes a polypeptide comprising an amino acid sequence that is at least 90% identical to a sequence selected from the group consisting of SEQ ID NOS: 14-26. In an embodiment, the plant is a monocot. In an embodiment, the plant is a rice plant. In certain embodiments, the plant further comprises a heterologous nucleic acid sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

### BRIEF DESCRIPTION OF THE SEQUENCE LISTING

The disclosure can be more fully understood from the following detailed description and the accompanying Sequence Listing that form a part of this application, which are incorporated herein by reference.

The sequence descriptions summarize the Sequence Listing attached hereto, which is hereby incorporated by reference. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

TABLE 1
Sequence Listing Description

| SEQ ID NOS | Description |
|---|---|
| SEQ ID NO: 1 | DP1492 LOC_Os03g14669.2 Core histone H2A/H2B/H3/H4, |
| | putative, expressed; Nucleotide Sequence |
| SEQ ID NO: 2 | DP1564 LOC_Os07g49460.1 Response regulator receiver |
| | domain containing protein, expressed; Nucleotide Sequence |
| SEQ ID NO: 3 | DP2300-LOC_Os07g15770.1 CCT motif family protein, |
| | expressed; Nucleotide Sequence |
| SEQ ID NO: 4 | DP0830 LOC_Os01g68700.1 BHLH transcription factor, |
| | putative, expressed; Nucleotide Sequence |
| SEQ ID NO: 5 | DP1315- LOC_Os02g54140.1 hsp20/alpha crystallin family |
| | protein, putative, expressed; Nucleotide Sequence |
| SEQ ID NO: 6 | DP1722 -LOC_Os07g38730.1 -tubulin/FtsZ domain |
| | containing protein, putative, expressed; Nucleotide Sequence |
| SEQ ID NO: 7 | DP1856-LOC_Os03g60080.1-NAC domain-containing |
| | protein 67, putative, expressed; Nucleotide Sequence |
| SEQ ID NO: 8 | DP1249 -LOC_Os04g44440.1-TCP family transcription |
| | factor, putative, expressed; Nucleotide Sequence |
| SEQ ID NO: 9 | DP1103- LOC_Os11g43970- AAA-type ATPase family |
| | protein, putative, expressed; Nucleotide Sequence |
| SEQ ID NO: 10 | DP0896- LOC_Os07g49290.1- PHD finger family protein, |
| | putative, expressed; Nucleotide Sequence |
| SEQ ID NO: 11 | DP0885 LOC_Os02g02970.1- expressed protein; Nucleotide |
| | Sequence |
| SEQ ID NO: 12 | DP0998 LOC_Os07g36600.1 universal stress protein domain |
| | containing protein, putative, expressed; Nucleotide Sequence |
| SEQ ID NO: 13 | DP0995 LOC_Os05g33550 methyl-binding domain protein |
| | MBD; Nucleotide Sequence |
| SEQ ID NO: 14 | DP1492 LOC_Os03g14669.2 Core histone H2A/H2B/H3/H4, |
| | putative, expressed; Amino Acid Sequence |
| SEQ ID NO: 15 | DP1564 LOC_Os07g49460.1 Response regulator receiver |
| | domain containing protein, expressed; Amino Acid Sequence |
| SEQ ID NO: 16 | DP2300-LOC_Os07g15770.1 CCT motif family protein, |
| | expressed; Amino Acid Sequence |
| SEQ ID NO: 17 | DP0830 LOC_Os01g68700.1 BHLH transcription factor, |
| | putative, expressed; Amino Acid Sequence |
| SEQ ID NO: 18 | DP1315- LOC_Os02g54140.1 hsp20/alpha crystallin family |
| | protein, putative, expressed; Amino Acid Sequence |
| SEQ ID NO: 19 | DP1722 -LOC_Os07g38730.1 -tubulin/FtsZ domain |
| | containing protein, putative, expressed; Amino Acid |
| | Sequence |
| SEQ ID NO: 20 | DP1856-LOC_Os03g60080.1-NAC domain-containing |
| | protein 67, putative, expressed; Amino Acid Sequence |
| SEQ ID NO: 21 | DP1249 -LOC_Os04g44440.1-TCP family transcription |
| | factor, putative, expressed; Amino Acid Sequence |
| SEQ ID NO: 22 | DP1103- LOC_Os11g43970- AAA-type ATPase family |
| | protein, putative, expressed; Amino Acid Sequence |
| SEQ ID NO: 23 | DP0896- LOC_Os07g49290.1- PHD finger family protein, |
| | putative, expressed; Amino Acid Sequence |
| SEQ ID NO: 24 | DP0885 LOC_Os02g02970.1- expressed protein; Amino Acid |
| | Sequence |
| SEQ ID NO: 25 | DP0998 LOC_Os07g36600.1 universal stress protein domain |
| | containing protein, putative, expressed; Amino Acid |
| | Sequence |
| SEQ ID NO: 26 | DP0995 LOC_Os05g33550 methyl-binding domain protein |
| | MBD; Amino Acid Sequence |
| SEQ ID NO: 27 | DP1492_CR1 |
| SEQ ID NO: 28 | DP1492_CR2 |
| SEQ ID NO: 29 | DP1564_CR1 |
| SEQ ID NO: 30 | DP1564_CR2 |
| SEQ ID NO: 31 | DP2300_CR1 |
| SEQ ID NO: 32 | DP2300_CR2 |
| SEQ ID NO: 33 | DP0830_CR1 |
| SEQ ID NO: 34 | DP0830_CR2 |
| SEQ ID NO: 35 | DP1315_CR1 |
| SEQ ID NO: 36 | DP1315_CR2 |
| SEQ ID NO: 37 | DP1722_CR1 |
| SEQ ID NO: 38 | DP1722_CR2 |
| SEQ ID NO: 39 | DP1856_CR1 |
| SEQ ID NO: 40 | DP1856_CR3 |
| SEQ ID NO: 41 | DP1249_CR1 |
| SEQ ID NO: 42 | DP1249_CR2 |
| SEQ ID NO: 43 | DP1103_CR1 |
| SEQ ID NO: 44 | DP1103_CR2 |
| SEQ ID NO: 45 | DP0896_CR1 |
| SEQ ID NO: 46 | DP0896_CR2 |
| SEQ ID NO: 47 | DP0885_CR1 |
| SEQ ID NO: 48 | DP0885_CR2 |
| SEQ ID NO: 49 | DP0998_CR1 |
| SEQ ID NO: 50 | DP0998_CR2 |
| SEQ ID NO: 51 | DP0995_CR1 |
| SEQ ID NO: 52 | DP0995_CR2 |
| SEQ ID NO: 53 | DP1103_CR3 |
| SEQ ID NO: 54 | DP1103_CR4 |
| SEQ ID NO: 55 | DP0896_CR3 |
| SEQ ID NO: 56 | DP0896_CR4 |
| SEQ ID NO: 57 | DP0885_CR3 |
| SEQ ID NO: 58 | DP0885_CR4 |
| SEQ ID NO: 59 | DP0998_CR3 |
| SEQ ID NO: 60 | DP0998_CR4 |
| SEQ ID NO: 61 | DP0995_CR3 |
| SEQ ID NO: 62 | DP0995_CR4 |

### DETAILED DESCRIPTION

The disclosure of all patents, patent applications, and publications cited herein are incorporated by reference in their entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein, the transitional phrase “consisting essentially of” generally refers to a composition, method that includes materials, steps, features, components, or elements, in addition to those literally disclosed, provided that these additional materials, steps, features, components, or elements do not materially affect the basic and novel characteristic(s) of the claimed subject matter.

Provided herein are methods for modifying the maturity of a plant, such as a rice plant, comprising, consisting essentially of, or consisting of introducing one or more nucleotide modifications, through targeted DNA modification at a genomic locus of the plant.

As used herein, “modifying maturity,” “maturity modulation,” or the like, refers to any detectable change in the time for a modified plant to reach a stage of development, as compared to a control plant (e.g., a wild-type plant that does not comprise the DNA modification). For example, maturity can be measured as the number of days to first flowering, panicle emergence, or panicle initiation, or alternatively, maturity can be measured as the number of days to 50% flowering, panicle emergence, or panicle initiation. The stage of development at which maturity is measured is not particularly limited. However, in certain embodiments maturity is measured based on the number of days to first flowering, panicle emergence, or panicle initiation.

In the stages of rice plant maturity, panicle initiation (PI) (generally referred to as stage 4) is marked by the emergence of the panicle. Often, young panicles that emerge of the last node, is a cone-shaped organ visible if the stem is dissected. The cone may become visible about 10 days after it is formed, depending on the maturity duration of the rice variety. During the development of the reproductive phase, the number of grains in the panicle is usually determined. For example, in short-duration varieties, maximum tillering, inter-node elongation, and panicle initiation are generally exhibited simultaneously or within a short window. Depending on the duration of the varieties, such as for example, medium to long-duration, these stages occur in the order mentioned above. Timing of panicle initiation in rice may also be influenced by many factors, such as temperature and photoperiod to which certain varieties have adapted. Panicle initiation generally marks the beginning of the reproductive phase in rice plant and therefore is a measure of maturity when evaluating rice plants.

Panicle development generally referred to as stage 5 in rice development, is characterized by the swelling of the bottom of the panicle leaf due to the panicle growing upwards inside the stem. After panicle initiation (stage 4), the panicle grows towards the top of the stem, causing a swelling in the stem called elongation. The organs of the flower develop, and the panicle grows until it reaches its final size before appearing from the flag leaf, generally referred to as heading.

Heading and flowering, generally referred to as stage 6, is characterized by the emergence of the panicle from the bottom of the panicle/flag leaf, which may take about two to three weeks to emerge from the stem fully. Three days after heading, flowering occurs, and the process continues until the panicle has completely appeared. Flowering generally means that the flower opens, and pollination takes place.

Milky stage, generally referred to as stage 7 occurs after fertilization and is characterized by swollen ovary, and the caryopsis develops until it reaches its mature size. The grain (caryopsis) is first aqueous and then reaches a milky consistency, observable when the grain is squeezed. The panicles are green and erect until this stage of rice reproductive development.

Dough stage, generally referred to as stage 8 is often characterized by softening of the grain and reaching a hard paste consistency after flowering. The erect panicle begins to droop along with the change in color of the grain that is characteristic of the variety e.g., yellow, red, black.

Maturity, generally referred to as stage 9 is often characterized by ripe grain and when the grant has reached its final size and maximum weight, characterized by the droopy appearance. Grains become hard and develop their characteristic colors. This stage is reached when about 85 to 90% of the panicle grains are ripe.

A “genomic locus of a plant” as used herein, generally refers to the location on a chromosome of the plant where a gene, such as a polynucleotide involved in flowering time regulation (FTR), is found. As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

A “regulatory element” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located.

An “enhancer” element is any nucleic acid molecule that increases transcription of a nucleic acid molecule when functionally linked to a promoter regardless of its relative position.

A “repressor” (also sometimes called herein silencer) is defined as any nucleic acid molecule which inhibits the transcription when functionally linked to a promoter regardless of relative position.

A “promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” generally refers to a product resulting from RNA polymerase-catalyzed transcription of a DNA sequence. When an RNA transcript is a perfect complimentary copy of a DNA sequence, it is referred to as a primary transcript or it may be a RNA sequence derived from posttranscriptional processing of a primary transcript and is referred to as a mature RNA. “Messenger RNA” (“mRNA”) generally refers to RNA that is without introns and that can be translated into protein by the cell. “cDNA” generally refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA generally refers to RNA transcript that includes mRNA and so can be translated into protein within a cell or in vitro. “Antisense RNA” generally refers to a RNA transcript that is complementary to all or part of a target primary transcript or mRNA and that blocks expression or transcripts accumulation of a target gene. The complementarity of an antisense RNA may be with any part of the specific gene transcript, i.e. at the 5′ non-coding sequence, 3′ non-coding sequence, introns, or the coding sequence. “Functional RNA” generally refers to antisense RNA, ribozyme RNA, or other RNA that may not be translated but yet has an effect on cellular processes.

“Targeted DNA modification” can be used synonymously with targeted DNA mutation and refers to the introduction of a site specification modification that alters or changes the nucleotide sequence at a specific genomic locus of the plant (e.g., rice).

In certain embodiments, the targeted DNA modification occurs at a genomic locus that comprises a polynucleotide involved in flowering time regulation (FTR).

In certain embodiments the polynucleotide involved in FTR is a polynucleotide that encodes a response regulator receiver domain containing protein, a CCT motif containing protein, BHLH transcription factor, TCP family transcription factor, NAC domain-containing protein, tubulin/FtsZ domain containing protein, hsp20/alpha crystallin protein, core histone H2A/H2B/H3/H4 putative protein, AAA-type ATPase family protein, universal stress protein domain containing protein, PHD finger family protein, and/or a methyl-binding domain protein.

The polynucleotide sequences, encoded proteins, and the corresponding genomic loci of the polynucleotides involved in FTR are known in the art or can be readily identified using routine methods in the art. In certain embodiments, the polynucleotide involved in FTR encodes a polypeptide comprising an amino acid sequence that is at least 90% (e.g., 91%, 92%, 93%, 94%, 95%, 96%, 97%, 98%, 99%, or 100%) identical to an amino acid sequence selected from the group consisting of SEQ ID NOs: 14-26.

Sequence alignments and percent identity calculations may be determined using a variety of comparison methods designed to detect similar or identical sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CAB/OS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CAB/OS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

In one embodiment the % sequence identity is determined over the entire length of the molecule (nucleotide or amino acid).

The targeted DNA modification described herein may be any modification known in the art such as, for example, insertion, deletion, single nucleotide polymorphism (SNP), and or a polynucleotide modification. Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region.

The type and location of the targeted DNA modification of the FTP polynucleotide is not particularly limited so long as the targeted DNA modification results in reduced expression or activity of the protein encoded by the FTR polynucleotide. In certain embodiments the targeted DNA modification is a deletion of one or more nucleotides, preferably contiguous, of the genomic locus.

As used herein “reduced,” “reduction,” or the like refers to any detectable decrease in an experimental group (e.g., rice plant with a targeted DNA modification described herein) as compared to a control group (e.g., wild-type rice plant that does not comprise the targeted DNA modification).

Accordingly, reduced expression of a protein comprises any detectable decrease in the total level of the protein in a sample and can be determined using routine methods in the art such as, for example, Western blotting and ELISA.

In certain embodiments, a reduction in the expression or activity of the protein encoded by the FTR polynucleotide is due to a targeted DNA modification at a genomic locus of a plant that results in one or more of the following: (a) reduced expression of the FTR polynucleotide; (b) reduced transcriptional activity of the protein encoded by the FTR polynucleotide; (c) generation of one or more alternatively spliced transcripts of the FTR polynucleotide; (d) deletion of one or more DNA binding domains of the encoded FTR polypeptide; (e) frameshift mutation in one or more exons of the FTR polynucleotide; (f) deletion of a substantial portion of the FTR polynucleotide or deletion of the full open reading frame of the FTR polynucleotide; (g) repression of an enhance motif present within a regulatory region encoding the FTR polynucleotide; or (h) modification of one or more nucleotides or deletion of a regulatory element operably linked to the expression of the FTR polynucleotide wherein the regulatory element is present within a promoter, intron, 3′UTR, terminator or a combination thereof.

In certain embodiments, the targeted DNA modification at a genomic locus involved in FTR results in plants (e.g., rice) that exhibit early maturity compared to control plants. For example, plants exhibiting early flowering compared to control plants. In certain embodiments, the modified plant's first flowing occurs in a range of about 5 to 15 days (e.g., 5 to 14, 5 to 13, 5 to 12, 5 to 11, 5 to 10, 5 to 9, 5 to 8, 5 to 7, 5 to 6) earlier than a control plant. In certain embodiments, the targeted DNA modification that results in early maturity occurs at a genomic locus comprising a polynucleotide involved in FTR encoding a response regulator receiver domain containing protein, a CCT motif containing protein, BHLH transcription factor, TCP family transcription factor, NAC domain-containing protein, tubulin/FtsZ domain containing protein, hsp20/alpha crystallin protein, and/or core histone H2A/H2B/H3/H4 putative protein. In certain embodiments, the DNA modification that results in early maturity is a deletion at the genomic locus of the polynucleotide involved in FTR.

In other embodiments, the targeted DNA modification results in plants (e.g., rice) that exhibit delayed maturity compared to control plants. For example, plants exhibiting a delayed first flowering time compared to control plant. In certain embodiments, the maturity of the plant is reduced by about 5%-50% compared to a control plant. In other words, the modified plant takes about 5%-50% longer (e.g., numbers of days) to reach a particular stage of development, as compared to a control plant. In certain embodiments, the targeted DNA modification that results in delayed maturity occurs at a genomic locus comprising a polynucleotide involved in FTR encoding AAA-type ATPase family protein, universal stress protein domain containing protein, PHD finger family protein, or a methyl-binding domain protein. In certain embodiments, the DNA modification that results in delayed maturity is a deletion at the genomic locus of the polynucleotide involved in FTR.

In certain embodiments, the genomic locus has more than one (e.g., 2, 3, 4, 5, 6, 7, 8, 9, or 10) targeted DNA modification. For example, the translated region and a regulatory element of a genomic locus may each comprise a targeted DNA modification.

In certain embodiments, the plant may have targeted DNA modifications at more than one (e.g., 2, 3, 4, 5, 6, 7, 8, 9, or 10) genomic loci that are involved in flowering time regulation of the plant (e.g., rice).

In certain embodiments of the methods described herein, the DNA modification(s) that modifies the maturity of the plant does not adversely affect other agronomic traits. For example is certain embodiments, the targeted DNA modification at a genomic locus comprising a polynucleotide involved in FTR does not: (a) significantly reduce grain yield, as measured by per rice plant or as a population of rice plants per unit area, (b) significantly reduce stature, as measured by a reduction in plant height, and/or (c) does not significantly alter root architecture, and/or root lodging compared to a control plant that does not comprise the modification.

The targeted DNA modification of the genomic locus may be done using any genome modification technique known in the art. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or Pl- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9 and Cpf1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide of the methods and compositions described herein may be any polynucleotide sequence that targets the genomic loci of a plant cell comprising a polynucleotide that encodes an amino acid sequence that is at least 90% (e.g., 91%, 92%, 93%, 94%, 95%, 96%, 97%, 98%, 99%, or 100%) identical to a sequence selected from the group consisting of SEQ ID NOs: 14-26. In certain embodiments, the guide polynucleotide is a guide RNA. The guide polynucleotide may also be present in a recombinant DNA construct.

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide an guidepolynucleotide/Cas endonuclease complex to a unique DNA target site.

The terms “knock-out”, “gene knock-out” and “genetic knock-out” are used interchangeably herein. A knock-out represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas endonuclease. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site of a Cas endonuclease. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Methods for transforming dicots, primarily by use of Agrobacterium tumefaciens, and obtaining transgenic plants have been published, among others, for cotton (U.S. Pat. Nos. 5,004,863, 5,159,135); soybean (U.S. Pat. Nos. 5,569,834, 5,416,011); Brassica (U.S. Pat. No. 5,463,174); peanut (Cheng et al., Plant Cell Rep. 15:653-657 (1996), McKently et al., Plant Cell Rep. 14:699-703 (1995)); Papaya (Ling et al., Bio/technology 9:752-758 (1991)); and pea (Grant et al., Plant Cell Rep. 15:254-258 (1995)). For a review of other commonly used methods of plant transformation see Newell, C. A., Mol. Biotechnol. 16:53-65 (2000). One of these methods of transformation uses Agrobacterium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U.S.A. 84:3962-3966 (1987)), microinjection, or particle bombardment (McCabe et al., Biotechnology 6:923-926 (1988); Christou et al., Plant Physiol. 87:671-674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, Calif., 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

Recombinant DNA constructs comprising one or more of the polynucleotide sequences set forth in SEQ ID NOs: 14-26 are provided herein.

Also provided are plants, plant cells, and/or seeds introduced with a polynucleotide described herein. In certain embodiments the plant, plant cell, or seed comprises a recombinant DNA construct comprising one or more of the polynucleotide sequences set forth in SEQ ID NOs: 14-26. In certain embodiments, the plant, plant cell, or seed comprises a recombinant DNA construct comprising one or more guide polynucleotides that target the genomic loci of a plant cell comprising a polynucleotide that encodes an amino acid sequence that is at least 90% (e.g., 91%, 92%, 93%, 94%, 95%, 96%, 97%, 98%, 99%, or 100%) identical to a sequence selected from the group consisting of SEQ ID NOs: 14-26.

The polynucleotide of the plant, plant cell, or seed can be stably introduced or can be transiently expressed by the plant, plant cell, or seed. In certain embodiments, the polynucleotide is stably introduced into the plant, plant cell, or seed.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “recombinant DNA construct” or “recombinant expression construct” is used interchangeably and generally refers to a discrete polynucleotide into which a nucleic acid sequence or fragment can be moved. Preferably, it is a plasmid vector or a fragment thereof comprising the promoters of the present disclosure. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The promoters for use in the vector may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Core promoters are often modified to produce artificial, chimeric, or hybrid promoters, and can further be used in combination with other regulatory elements, such as cis-elements, 5′UTRs, enhancers, or introns, that are either heterologous to an active core promoter or combined with its own partial or complete regulatory elements. In certain embodiments the promoter of the recombinant DNA construct may be a tissue-specific promoter, developmentally regulated promoter, or a constitutive promoter.

“Tissue-specific promoter” and “tissue-preferred promoter” are used interchangeably to refer to a promoter that is expressed predominantly but not necessarily exclusively in one tissue or organ, but that may also be expressed in one specific cell. “Developmentally regulated promoter” generally refers to a promoter whose activity is determined by developmental events. “Constitutive promoter” generally refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

In certain embodiments the promoter of the recombinant DNA construct is heterologous to the expressed nucleotide sequence. A “heterologous nucleotide sequence” generally refers to a sequence that is not naturally occurring with the sequence of the disclosure. While this nucleotide sequence is heterologous to the sequence, it may be homologous, or native, or heterologous, or foreign, to the plant host. However, it is recognized that the instant sequences may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

The isolated promoter sequence comprised in the recombinant DNA construct of the present disclosure can be modified to provide a range of constitutive expression levels of the heterologous nucleotide sequence. Thus, less than the entire promoter regions may be utilized and the ability to drive expression of the coding sequence retained. However, it is recognized that expression levels of the mRNA may be decreased with deletions of portions of the promoter sequences. Likewise, the tissue-independent, constitutive nature of expression may be changed.

Modifications of the isolated promoter sequences of the present disclosure can provide for a range of constitutive expression of the heterologous nucleotide sequence. Thus, they may be modified to be weak constitutive promoters or strong constitutive promoters. Generally, by “weak promoter” is intended a promoter that drives expression of a coding sequence at a low level. By “low level” is intended levels about 1/10,000 transcripts to about 1/100,000 transcripts to about 1/500,000 transcripts. Conversely, a strong promoter drives expression of a coding sequence at high level, or at about 1/10 transcripts to about 1/100 transcripts to about 1/1,000 transcripts. Similarly, a “moderate constitutive” promoter is somewhat weaker than a strong constitutive promoter like the maize ubiquitin promoter.

The term “operably linked” or “functionally linked” generally refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “initiate transcription”, “initiate expression”, “drive transcription”, and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “expression”, as used herein, generally refers to the production of a functional end-product e.g., an mRNA or a protein (precursor or mature).

The term “expression cassette” as used herein, generally refers to a discrete nucleic acid fragment into which a nucleic acid sequence or fragment can be cloned or synthesized through molecular biology techniques.

“Transformation” as used herein generally refers to both stable transformation and transient transformation. “Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” generally refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The heterologous polynucleotide can be stably integrated within the genome such that the polynucleotide is passed on to successive generations. The heterologous polynucleotide may be integrated into the genome alone or as part of a recombinant DNA construct. The alterations of the genome (chromosomal or extra-chromosomal) by conventional plant breeding methods, by genome editing procedures that do not result in an insertion of a foreign polynucleotide, or by naturally occurring events such as random cross-fertilization, non-recombinant viral infection, non-recombinant bacterial transformation, non-recombinant transposition, or spontaneous mutation are also methods of modifying a host genome.

“Transient expression” generally refers to the temporary expression of often reporter genes such as β-glucuronidase (GUS), fluorescent protein genes ZS-GREEN1, ZS-YELLOW1 N1, AM-CYAN1, DS-RED in selected certain cell types of the host organism in which the transgenic gene is introduced temporally by a transformation method. The transformed materials of the host organism are subsequently discarded after the transient gene expression assay.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

The plant, plant cells, and seeds of the compositions and methods described herein are not particularly limited and may be any plant species including, but not limited to, monocots and dicots. The terms “monocot” and “monocotyledonous plant” are used interchangeably herein. A monocot of the current disclosure includes rice. The terms “dicot” and “dicotyledonous plant” are used interchangeably herein. A dicot of the current disclosure includes the following families: Brassicaceae, Leguminosae, and Solanaceae.

In certain embodiments the plant is a rice plant of the genus Oryza. In certain embodiments, the rice plant is Oryza sativa, optionally of the variety indicia, or Oryza glaberrima. In certain embodiments, the rice plant is an inbred rice line (e.g., female inbred line), while in other embodiments the rice plant is a hybrid rice plant.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The plant, plant cell, and seed, of the compositions and methods described herein may further comprise a heterologous nucleic acid sequence that confers advantageous properties, such as improved agronomics, to the plant, plant cell, and/or seed. The heterologous nucleic acid sequences are known to those of ordinary skill in the art, and can be routinely incorporated in the plant, plant cell, and/or seeds described herein using routine methods in the art, such as those described herein.

In certain embodiments the heterologous nucleic acid sequence is selected from the group consisting of a reporter gene, a selection marker, a disease resistance gene, a herbicide resistance gene, an insect resistance gene, a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

In certain embodiments, the present disclosure contemplates the transformation of a recipient cell with more than one advantageous gene. Two or more genes can be supplied in a single transformation event using either distinct gene-encoding vectors, or a single vector incorporating two or more gene coding sequences. Any two or more genes of any description, such as those conferring herbicide, insect, disease (viral, bacterial, fungal, and nematode), or drought resistance, oil quantity and quality, or those increasing yield or nutritional quality may be employed as desired.

### EXAMPLES

The present disclosure is further defined in the following Examples. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Rice Gene Targets for Maturity Modulation

This example demonstrates the identification of rice gene targets for maturity modification.

Optimal flowering time is a critical factor that determines crop yield and hybrid seed production. Maturity can be generally described as time from seedling to harvest, change in flowering time, either early or late influence the maturity of the plants. The modulation of flowering time to growing conditions is not possible without a better understanding of genes involved in flowering time and their regulation.

To identify genes involved in rice FTR, rice activation tag populations were screened in field conditions and analyzed for the flowering phenotype. Lines showing either late or early flowering compared to control were identified, and genes present both upstream and downstream to the T-DNA insertion site in the genomic DNA were cloned and overexpressed under a constitutive promoter. The genes which gave an early or late flowering phenotype upon overexpression were selected to reverse the phenotype through a CRISPR-Cas mediated SDN1 genome editing approach.

### Example 2

### Targeted DNA Modification of Genomic Loci Involved in Flowering Time Regulation

Targeted DNA modification of the genomic loci of the genes identified Example 1 was performed to determine the effect on maturity.

CRISPR/Cas9 assisted targeted genome editing offers to knock-out any gene sequence and generate knock-outs through small deletions, internal small fragment deletions within the target gene or full-length gene deletion. Through CRISPR-Cas genome editing, new variations of the alleles are introduced directly in the elite target germplasm with minimal genetic drag associated with conventional breeding material. The target genes include DP1492, DP1249, DP1856, DP1722, DP1315, DP0830, DP2300, DP1564, DP1103, DP0998, DP0885, DP0896 and DP0995. Tables 1-5 below provide the guide polynucleotides and targeting strategies to knock-down expression of the target genes.

TABLE 1
CRISPR/Cas Targeted Sequences of Polynucleotides Involved in FTR

| Target | | Target |
|---|---|---|
| Sequence | | Sequence SEQ |
| Designation | Target Sequence (5′-3′) | ID NO |
| DP1492_CR1 | AGCAGCGCCGGATGTGGACCAGGG | SEQ ID NO: 27 |
| DP1492_CR2 | AACCAGCAGCTACCCTACGCCGG | SEQ ID NO: 28 |
| DP1564_CR1 | AACGACGCCGTGCCTGGGGCTGGG | SEQ ID NO: 29 |
| DP1564_CR2 | CCGGATGGCGGACCAACCTCCGGT | SEQ ID NO: 30 |
| | (target cut site is on the complimentary strand) | |
| DP2300_CR1 | ATGAGGAGTCGCCAAATTATCAGG | SEQ ID NO: 31 |
| DP2300_CR2 | CCGGCGGGCTCACGTTCGACGTCT | SEQ ID NO: 32 |
| | (target cut site is on the complimentary strand) | |
| DP0830_CR1 | CCAAGGGGGCTTCCGACGGCAAT (target | SEQ ID NO: 33 |
| | cut site is on the complimentary strand) | |
| DP0830_CR2 | AGGAGAATTCCGGCAAGGGG | SEQ ID NO: 34 |
| DP1315_CR1 | AGCCCAAGTTGGTAGCCATGACGG | SEQ ID NO: 35 |
| DP1315_CR2 | CCAAGTCCGACATCCAGGTGCGT (target | SEQ ID NO: 36 |
| | cut site is on the complimentary strand) | |
| DP1722_CR1 | ATCGGCCAGGCCGGGATCCAGG | SEQ ID NO: 37 |
| DP1722_CR2 | ACCCTGAGCAGCTCATCTCTGGG | SEQ ID NO: 38 |
| DP1856_CR1 | ATCTCTACAAGTTCGACCCGTGG | SEQ ID NO: 39 |
| DP1856_CR3 | ACTCGCACACCCACTCGTGGGG | SEQ ID NO: 40 |
| DP1249_CR1 | ACCTTCCAGGTCTACCGGCCCATGG | SEQ ID NO: 41 |
| DP1249_CR2 | AGCTCACCCGCGAGCTGGGG | SEQ ID NO: 42 |
| DP1103_CR1 | CCTATCAGTGTTCCTCAGTTTTCT (target | SEQ ID NO: 43 |
| | cut site is on the complimentary strand) | |
| DP1103_CR2 | CCAGAGTCGCAGAAAGATGCT (target cut | SEQ ID NO: 44 |
| | site is on the complimentary strand) | |
| DP0896_CR1 | CCTTGCGCCCAGGCTCCGCTACGT | SEQ ID NO: 45 |
| | (target cut site is on the complimentary strand) | |
| DP0896_CR2 | AGCTCATCTACACCACGAAGCGAGG | SEQ ID NO: 46 |
| DP0885_CR1 | ATGGTGGACATCGTGGCTTCGAGCGG | SEQ ID NO: 47 |
| DP0885_CR2 | CCTTCGGAAATTCGCATAGCGAT (target | SEQ ID NO: 48 |
| | cut site is on the complimentary strand) | |
| DP0998_CR1 | CCGGAGTCCCCGGGGGTGTTCT (target | SEQ ID NO: 49 |
| | cut site is on the complimentary strand) | |
| DP0998_CR2 | ACCACTGTGTGTGCCCCGTGGTGG | SEQ ID NO: 50 |
| DP0995_CR1 | CCTGACTGCTCGTGTGAAGACCCT | SEQ ID NO: 51 |
| | (target cut site is on the complimentary strand) | |
| DP0995_CR2 | ACTGTTTCTCACAACTCCGTGTGG | SEQ ID NO: 52 |
| DP1103_CR3 | ATTCACACGTTACAAACGCACGGG | SEQ ID NO: 53 |
| DP1103_CR4 | ACCTTTGTGGTGCTTCCTTGTCTAGG | SEQ ID NO: 54 |
| DP0896_CR3 | ATCGGTTACGAATCAATCCTCTCGG | SEQ ID NO: 55 |
| DP0896_CR4 | GTGCGCTCTGTTCCTGGATTCGG | SEQ ID NO: 56 |
| DP0885_CR3 | AGAAGAAGGCGGATTCCTTTTTGG | SEQ ID NO: 57 |
| DP0885_CR4 | AAGAGGCCGGGAAACCTAGACCAGG | SEQ ID NO: 58 |
| DP0998_CR3 | ACTGCCCCACATGGTCCGCATTTGG | SEQ ID NO: 59 |
| DP0998_CR4 | TGTTTCTCATGCATGAAGGATAAGG | SEQ ID NO: 60 |
| DP0995_CR3 | GGCCCAGCAACGCGGCCCGAGG | SEQ ID NO: 61 |
| DP0995_CR4 | ATGGTTTTGAATCGATTGACCTGG | SEQ ID NO: 62 |

TABLE 2
Description of the Edits Observed in the Targeted FTR Genes

| | | Target | | | |
|---|---|---|---|---|---|
| | Target | Sequence | Relative | Relative | |
| | Sequence | SEQ ID | Start | End | |
| Gene | Designation | NO | Position (bp) | Position (bp) | Edit |
| DP1492 | DP1492_CR1 | SEQ ID | 2076 (based on | 2099 (based | Internal |
| full-length | | NO: 27 | SEQ ID NO: 1 | on SEQ ID | deletions |
| genomic | | | position) | NO: 1 | |
| sequence | | | | position) | |
| (SEQ ID | DP1492_CR2 | SEQ ID | 1462 | 1484 | Internal |
| NO: 1) | | NO: 28 | | | deletions |
| DP1564 | DP1564_CR1 | SEQ ID | 792 (based on | 815 (based | Internal |
| full-length | | NO: 29 | SEQ ID NO: 2 | on SEQ ID | deletions |
| genomic | | | position) | NO: 2 | |
| sequence | | | | position) | |
| (SEQ ID | DP1564_CR2 | SEQ ID | 834 | 857 | Internal |
| NO: 2) | | NO: 30 | | | deletions |
| DP2300 | DP2300_CR1 | SEQ ID | 14 (based on | 37 (based on | Internal |
| full-length | | NO: 31 | SEQ ID NO: 3 | SEQ ID NO: 3 | deletions |
| genomic | | | position) | position) | |
| sequence | DP2300_CR2 | SEQ ID | 697 | 720 | Internal |
| (SEQ ID | | NO: 32 | | | deletions |
| NO: 3) | | | | | |
| DP0830 | DP0830_CR1 | SEQ ID | 1136 (based on | 1158 (based | Internal |
| full-length | | NO: 33 | SEQ ID NO: 4 | on SEQ ID | deletions |
| genomic | | | position) | NO: 4 | |
| sequence | | | | position) | |
| (SEQ ID | DP0830_CR2 | SEQ ID | 1363 | 1382 | Internal |
| NO: 4) | | NO: 34 | | | deletions |
| DP1315 | DP1315_CR1 | SEQ ID | 40 (based on | 63 (based on | Internal |
| full-length | | NO: 35 | SEQ ID NO: 5 | SEQ ID NO: 5 | deletions |
| genomic | | | position) | position) | |
| sequence | DP1315_CR2 | SEQ ID | 283 | 305 | Internal |
| (SEQ ID | | NO: 36 | | | deletions |
| NO: 5) | | | | | |
| DP1722 | DP1722_CR1 | SEQ ID | 155 (based on | 176 (based | Internal |
| full-length | | NO: 37 | SEQ ID NO: 6 | on SEQ ID | deletions |
| genomic | | | position) | NO: 6 | |
| sequence | | | | position) | |
| (SEQ ID | DP1722_CR2 | SEQ ID | 1303 | 1325 | Internal |
| NO: 6) | | NO: 38 | | | deletions |
| DP1856 | DP1856_CR1 | SEQ ID | 238 (based on | 260 (based | Internal |
| full-length | | NO: 39 | SEQ ID NO: 7 | on SEQ ID | deletions |
| genomic | | | position) | NO: 7 | |
| sequence | | | | position) | |
| (SEQ ID | DP1856_CR3 | SEQ ID | 796 | 817 | Internal |
| NO: 7) | | NO: 40 | | | deletions |
| DP1249 | DP1249_CR1 | SEQ ID | 73 (based on | 97 (based on | Internal |
| full-length | | NO: 41 | SEQ ID NO: 8 | SEQ ID NO: 8 | deletions |
| genomic | | | position) | position) | |
| sequence | DP1249_CR2 | SEQ ID | 293 | 312 | Internal |
| (SEQ ID | | NO: 42 | | | deletions |
| NO: 8) | | | | | |
| DP1103 | DP1103_CR1 | SEQ ID | 2030 (based on | 2053 (based | Internal |
| full-length | | NO: 43 | SEQ ID NO: 9 | on SEQ ID | deletions |
| genomic | | | position) | NO: 9 | |
| sequence | | | | position) | |
| (SEQ ID | DP1103_CR2 | SEQ ID | 4020 | 4040 | Internal |
| NO: 9) | | NO: 44 | | | deletions |
| DP0896 | DP0896_CR1 | SEQ ID | 201 (based on | 224 (based | Internal |
| full-length | | NO: 45 | SEQ ID NO: 10 | on SEQ ID | deletions |
| genomic | | | position) | NO: 10 | |
| sequence | | | | position) | |
| (SEQ ID | DP0896_CR2 | SEQ ID | 896 | 920 | Internal |
| NO: 10) | | NO: 46 | | | deletions |
| DP0885 | DP0885_CR1 | SEQ ID | 381 (based on | 406 (based | Internal |
| full-length | | NO: 47 | SEQ ID NO: 11 | on SEQ ID | deletions |
| genomic | | | position) | NO: 11 | |
| sequence | | | | position) | |
| (SEQ ID | DP0885_CR2 | SEQ ID | 1048 | 1070 | Internal |
| NO: 11) | | NO: 48 | | | deletions |
| DP0998 | DP0998_CR1 | SEQ ID | 322 (based on | 343 (based | Internal |
| full-length | | NO: 49 | SEQ ID NO: 12 | on SEQ ID | deletions |
| genomic | | | position) | NO: 12 | |
| sequence | | | | position) | |
| (SEQ ID | DP0998_CR2 | SEQ ID | 857 | 880 | Internal |
| NO: 12) | | NO: | 50 | | deletions |
| DP0995 | DP0995_CR1 | SEQ ID | 1594 (based on | 1617 (based | Internal |
| full-length | | NO: 51 | SEQ ID NO: 13 | on SEQ ID | deletions |
| genomic | | | position) | NO: 13 | |
| sequence | | | | position) | |
| (SEQ ID | DP0995_CR2 | SEQ ID | 1873 | 1896 | Internal |
| NO: 13) | | NO: 52 | | | deletions |

TABLE 3
Description of Edits Expected in Variants Generated by Single Guide Constructs

| Strategy | Construct | Description of the edits |
|---|---|---|
| Single | DP1249-CR1 | Guide designed to create small deletions in exon; |
| guide | | observed 1-2 base pair deletions at the target cut site |
| | DP1249-CR2 | Guide designed to create small deletions in exon; |
| | DP1315_CR1 | Guide designed to create small deletions in exon 1; |
| | | observed 1-6 base pair deletion at the target cut site. |
| | DP1722_CR1 | Guide designed to create small deletion in exon 1; |
| | | observed single nucleotide deletions at the target cut |
| | | site. |
| | DP1722_CR2 | Guide designed to create small deletion in exon 2; |
| | | observed 1-3 base pair deletions at the target cut site. |
| | DP1856-CR1 | Guide designed to create small deletion in exon 1; |
| | | observed 1-3 base pair deletions at the target cut site. |
| | DP1856-CR3 | Guide designed to create small deletion in exon 2; |
| | | observed 2-6 base pair deletions at the target cut site. |
| | DP0830_CR1 | Guide designed to create small deletions in exon 1; |
| | | observed 1-4 base pair deletions at the target cut site. |
| | DP1492_CR1 | Guide designed to create small deletions in exon; |
| | | observed 2-6 base pair deletions at the target cut site |
| | DP1492_CR2 | Guide designed to create small deletions in exon |
| | DP2300_CR1 | Guide designed to create small deletion in exon 1; |
| | | observed 3-4 base pair deletions at the target cut site. |
| | DP2300_CR2 | Guide designed to create small deletion in exon 2 |
| | DP1564_CR1 | NA |
| | DP1564_CR2 | NA |

TABLE 4
Description of Edits Expected in Variants Generated by Dual Guide Constructs

| Strategy | Construct | Description of the edits |
|---|---|---|
| Dual | DP1249 CR1 + CR2 | Designed two guides in exon to delete an internal |
| guides | | fragment of ~215 bp in ordered to knock-out the |
| | | target gene, DP1249. |
| | DP1315 CR1 + CR2 | Designed two guides in exon 1 to delete an internal |
| | | fragment of ~231 bp in ordered to knock-out the |
| | | target gene, DP1315. |
| | DP1492 CR1 + CR2 | Designed two guides in exon to delete an internal |
| | | fragment of ~615 bp in ordered to knock-out the |
| | | target gene, DP1492. |
| | DP1564 CR1 + CR2 | Designed two guides in exon 1 to delete an internal |
| | | fragment of ~30 bp to knock-out the target gene, |
| | | DP1564. |
| | DP1722 CR1 + CR2 | Designed two guides one in exon 1 and another one |
| | | in exon 2 to delete an internal fragment of ~1149 to |
| | | knock-out the target gene, DP1722. |
| | DP1856 CR1 + CR3 | Designed two guides one in exon 1 and another one |
| | | in exon 2 to delete an internal fragment of ~557 bp to |
| | | knock-out the target gene, DP1856. |
| | DP2300 CR1 + CR2 | Designed two guides one in exon 1 and another one |
| | | in exon 2 to delete an internal fragment of ~671 bp to |
| | | knock-out the target gene, DP2300. |
| | DP0830 CR1 + CR2 | Designed two guides one in exon 1 and another one |
| | | in exon 2 to delete an internal fragment of ~322 bp to |
| | | knock-out the target gene, DP0830. |

TABLE 5
Description of Edits Expected in Variants Generated by Dual Guide Constructs

| Strategy | Construct | Description of the edits |
|---|---|---|
| Dual | DP1103 CR1 + CR2 | Designed two guides one in Exon 4 and one in exon |
| guides | | 10 to delete an internal fragment of 1990 bp to |
| | | knock out the target gene, DP1103. |
| | DP0896 CR1 + CR2 | Designed two guides in Exon 1 to delete ~708 bp |
| | | fragment to knock out the target gene, DP0896. |
| | DP0885 CR1 + CR2 | Designed two guides one in Exon 1 and another |
| | | one in exon 2 to delete ~827 bp fragment in order to |
| | | knock-out the target gene, DP0885. |
| | DP0998 CR1 + CR2 | Designed two guides to delete an internal fragment |
| | | of ~550 bp to knock-out the target gene, DP0998. |
| | DP0995 CR1 + CR2 | Designed two guides in Exon 2 to delete ~279 bp |
| | | fragment to knock out the target gene, DP0995 |
| | DP1103 CR3 + CR4 | Designed two guides one in upstream to the |
| | | promoter region and another one in terminator |
| | | region to delete 13584 bp fragment |
| | DP0896 CR3 + CR4 | Designed two guides one in promoter and another |
| | | one in terminator region to delete 3512 bp fragment |
| | DP0885 CR3 + CR4 | Designed two guides one in promoter and another |
| | | one in terminator region to delete 3516 bp fragment |
| | DP0998 CR3 + CR4 | Designed two guides one in upstream to the |
| | | promoter and another one in downstream to the |
| | | terminator region to delete 7152 bp fragment |
| | DP0995 CR3 + CR4 | Designed two guides one in promoter region and |
| | | another one in downstream to the terminator region |
| | | to delete 2992 bp fragment |

### Example 3

### Phenotypic Analysis of Rice Plants with Maturity Modulation

Rice Transformation

The method used to generate genome edited variants through gene gun mediated particle bombardment is described below:

1) Seed sterilization and Callus induction: Seeds from rice inbred lines; IRV95, SDIA18G9C and SRPA17M5C were sterilized in 75% ethanol for 2-3 minutes and washed thoroughly with water and incubated in 4% sodium hypochlorite for 10 minutes. The seeds were then washed 5 times with water and dried completely at room temperature. The dried seeds were inoculated on callus inducing media and the plates were incubated at 28° C. in light for 5-7 days. After that the proliferating calli obtained from rice seeds were placed on osmotic media for 4 hours before being bombarded with DNA:gold particles.

2) Particle Bombardment:

a. Preparation of gold micro carriers: Sufficient amount of gold particles (amount of gold particles depends on the number of bombardments) were weighed and placed in 2.0 ml eppendorf tubes. One ml of 100% ethanol was added to the tube and sonicated for 30 sec before centrifuging for 1 min. The pellet containing the gold particles was resuspended in 1 ml of 100% ethanol, vortexed for 30 seconds and centrifuged again. This step was repeated twice before resuspending the pellet in 1 ml of sterile water. Fifty μl of gold particle suspension was aliquoted into eppendorf tubes and stored at 4° C.

b. DNA and gold particle preparation: Five μg of DNA, 50 μl of 2.5 mM CaCl₂) and 20 μl of 0.1 M spermidine were added to 50 μl of gold particle suspension; vortexed for 1-2 minutes and allowed the mixture to settle down for 5 minutes. The tubes were centrifuged for 2 minutes before discarding the supernatant. The pellet was resuspended in 40 μl of 100% ethanol and mixed gently by vortexing and 5 μl of sample was quickly dispensed onto macrocarrier disks and dried completely.

c. Particle bombardment using Bio-Rad gene gun (PDS 1000): Macro carrier disk carrying DNA:gold particle prep were loaded onto macro carrier disk holder and stopping screen was placed on top of the disk. Manufacturer's instructions were followed to deliver DNA:gold particles onto tissue samples which were placed on osmotic medium. After bombardment, the tissue samples were kept on the same osmotic medium for 24 h at 32° C. in dark.

3) Selection and regeneration of transformed variants: After 24 hours post bombardment, the samples were sub-cultured on to resting media and kept in dark at 28° C. for 5 days. The cultures were then transferred to selection media containing Hygromycin as selectable agent. After 3-4 selection cycles, proliferating, hygromycin resistant and Zs-Yellow positive callus variants were sub-cultured onto regeneration media and then to rooting and hardening media to obtain stable lines. Each independent line was transferred to an individual pot in greenhouse and samples were collected to perform molecular and phenotypic analysis.

### Phenotypic Analysis

Flowering generally means that the flower opens, and pollination takes place. Days to 50% flowering (DFF) refers to number of days when 50% of the panicles reach the flowering state. The results of the phenotypic analysis of the T0 or T1 population are provided in Tables 6-8.

TABLE 6
Flowering Data Collected from T0 Variants

| | Target | Target | Days to 50% |
|---|---|---|---|
| | Sequence | Sequence | flowering |
| Gene | Designation | SEQ ID NO | (DFF) |
| DP1492 full-length | DP1492_CR1 | SEQ ID NO: 27 | 66-86 |
| genomic sequence | DP1492_CR2 | SEQ ID NO: 28 | NA |
| (SEQ ID NO: 1) | | | |
| DP1564 full-length | DP1564_CR1 | SEQ ID NO: 29 | NA |
| genomic sequence | DP1564_CR2 | SEQ ID NO: 30 | NA |
| (SEQ ID NO: 2) | | | |
| DP2300 full-length | DP2300_CR1 | SEQ ID NO: 31 | NA |
| genomic sequence | DP2300_CR2 | SEQ ID NO: 32 | NA |
| (SEQ ID NO: 3) | | | |
| DP0830 full-length | DP0830_CR1 | SEQ ID NO: 33 | 54-66 |
| genomic sequence | DP0830_CR2 | SEQ ID NO: 34 | NA |
| (SEQ ID NO: 4) | | | |
| DP1315 full-length | DP1315_CR1 | SEQ ID NO: 35 | 51-68 |
| genomic sequence | DP1315_CR2 | SEQ ID NO: 36 | NA |
| (SEQ ID NO: 5) | | | |
| DP1722 full-length | DP1722_CR1 | SEQ ID NO: 37 | 58-69 |
| genomic sequence | DP1722_CR2 | SEQ ID NO: 38 | 59-76 |
| (SEQ ID NO: 6) | | | |
| DP1856 full-length | DP1856_CR1 | SEQ ID NO: 39 | 56-67 |
| genomic sequence | DP1856_CR3 | SEQ ID NO: 40 | 57-70 |
| (SEQ ID NO: 7) | | | |
| DP1249 full-length | DP1249_CR1 | SEQ ID NO: 41 | 58-68 |
| genomic sequence | DP1249_CR2 | SEQ ID NO: 42 | NA |
| (SEQ ID NO: 8) | | | |
| DP1103 full-length | DP1103_CR1 | SEQ ID NO: 43 | In the process |
| genomic sequence | DP1103_CR2 | SEQ ID NO: 44 | of generating |
| (SEQ ID NO: 9) | | | variants |
| DP0896 full-length | DP0896_CR1 | SEQ ID NO: 45 | Generating |
| genomic sequence | DP0896_CR2 | SEQ ID NO: 46 | variants |
| (SEQ ID NO: 10) | | | |
| DP0885 full-length | DP0885_CR1 | SEQ ID NO: 47 | Generating |
| genomic sequence | DP0885_CR2 | SEQ ID NO: 48 | variants |
| (SEQ ID NO: 11) | | | |
| DP0998 full-length | DP0998_CR1 | SEQ ID NO: 49 | Generating |
| genomic sequence | DP0998_CR2 | SEQ ID NO: 50 | variants |
| (SEQ ID NO: 12) | | | |
| DP0995 full-length | DP0995_CR1 | SEQ ID NO: 51 | Generating |
| genomic sequence | DP0995_CR2 | SEQ ID NO: 52 | variants |
| (SEQ ID NO: 13) | | | |

TABLE 7
Flowering Data Collected from T0 Variants

| | | | Days to 50% |
|---|---|---|---|
| Approach | Target sequences | Target SEQ ID NO | flowering |
| Dual Guide | DP1492 CR1 + CR2 | SEQ ID NOs: 27 + 28 | 60-74 |
| | DP1564 CR1 + CR2 | SEQ ID NOs: 29 + 30 | 57-80 |
| | DP2300 CR1 + CR2 | SEQ ID NOs: 31 + 32 | 65-78 |
| | DP1315 CR1 + CR2 | SEQ ID NOs: 35 + 36 | 67-68 |
| | DP1722 CR1 + CR2 | SEQ ID NOs: 37 + 38 | 72-89 |
| | DP1856 CR1 + CR3 | SEQ ID NOs: 39 + 40 | 74-84 |
| | DP1249 CR1 + CR2 | SEQ ID NOs: 41 + 42 | 63-72 |
| | DP0830 CR1 + CR2 | SEQ ID NOs 33 + 34 | 79-90 |

TABLE 8
Flowering Data Collected from T0 Variants

| | Target | Target | | | | |
|---|---|---|---|---|---|---|
| | sequence | sequence | | No of | Days to 50% flowering | |
| Gene | designation | ID No | | mutants | Range | Average |
| DP0998 | DP0998 | Sequence ID Nos | Mutants | 7 | 106-121 | 113 |
| | CR3 + CR4 | 59 + 60 | NGS-WT | 8 | 70-112 | 86 |
| DP0896 | DP0896 | Sequence ID Nos | Mutants | 8 | 88-108 | 102 |
| | CR3 + CR4 | 55 + 56 | NGS-WT | 5 | 69-100 | 88 |
| DP0885 | DP0885 | Sequence ID Nos | Mutants | 3 | 77-100 | 85 |
| | CR3 + CR4 | 57 + 58 | NGS-WT | 7 | 79-87 | 83 |
| DP1103 | DP1103 | Sequence ID Nos | In the process of generating variants | | | |
| | CR3 + CR4 | 53 + 54 | | | | |
| DP0995 | DP0995 | Sequence ID Nos | In the process of generating variants | | | |
| | CR3 + CR4 | 61 + 62 | | | | |
| IRV95 | | Seed derived | | 3 | 84-94 | 88 |
| | | wild-type | | | | |

The results of these studies show that plant maturity can be shortened by modulating flowering time phenotype of plants through modulation of putative Core Histone H2A/H2B/H3/H4 gene (DP1492); response regulator receiver domain containing protein (DP1564); CCT motif family protein (DP2300); Putative BHLH transcription factor (DP0830); putative TCP family transcription factor (DP1249); putative NAC domain-containing protein 67 (DP1856); tubulin/FtsZ domain containing protein gene (DP1722) or putative hsp20/alpha crystallin family protein gene (DP1315). Phenotype upon knock-out of genes listed resulted in early flowering and shortened maturity with no known pleotropic effects as compared to wild-type control rice plants. The guide RNA/Cas9 endonuclease system is used to target and induce a double strand break at a Cas9 endonuclease target site located within the coding region of the genes listed. Plants comprising small deletions or internal fragment deletions within the coding region of the genes were selected and evaluated for the shortened maturity phenotype.

Conversely, plant maturity can be delayed by modulating flowering time phenotype of plants through modulation of Universal stress protein domain containing protein (DP0998); putative AAA-type ATPase family protein gene (DP1103); putative PHD finger family protein gene (DP0896); methyl-binding domain protein gene (DP0995) or ‘expressed protein’ gene (DP0885) gene. Phenotype upon knock-out of genes listed will result in delayed flowering and eventually delayed maturity with no known pleotropic effects as compared to wild-type control rice plants. The guide RNA/Cas9 endonuclease system is used to target and induce a double strand break at a Cas9 endonuclease target site located within the coding region of the genes listed. Plants comprising deletion of few nucleotides or a small internal fragment within the coding region of the genes will be selected and evaluated for the late maturity phenotype.

### Example 4

### Transgenic Plants Overexpressing Genes Involved in Flowering Time Regulation

Transgenic plants were generated by transforming constructs carrying the indicated gene (SEQ ID NOs 9, 10, 11, 12, 13) cloned under maize ubiquitin promoter and PINII terminator. Events generated were evaluated in greenhouse (T0) and net house (T1) conditions. Days to 50% flowering data was collected from all the T0 events generated and compared with the data collected from controls. T1 lines derived from T0 events which were transformed with either DP1103, DP0998 or DP0995 gene constructs were evaluated and collected both days to 50% flowering and days to maturity. Data collected from T1 lines, Table 9, overexpressing DP0995 gene under maize ubiquitin promoter showed 10-12 day earliness in flowering and maturity compared to seed derived wild-type plants.

TABLE 9
Flowering Data Collected from T1 Transgenic Rice Events

| | Sequence | T0 event | No of T1 | Days to 50% | | Days to |
|---|---|---|---|---|---|---|
| Gene | ID No | ID | lines | flowering | Average | Maturity |
| DP0995 | SEQ ID | 321798813 | 10 | 74-79 | 76 | 106 |
| | NO: 13 | 321798820 | 10 | 76-82 | 78 | 108 |
| | | 321798822 | 10 | 79-84 | 81 | 111 |
| | | 321798824 | 10 | 78-82 | 79 | 109 |
| | | 321798826 | 10 | 77-83 | 81 | 121 |
| DP0998 | SEQ ID | 318816819 | 10 | 87-92 | 90 | 120 |
| | NO: 12 | 318816825 | 10 | 89-92 | 91 | 121 |
| | | 318816829 | 10 | 86-95 | 92 | 122 |
| DP1103 | SEQ ID | 320601056 | 10 | 92 | 92 | 122 |
| | NO: 9 | 320601058 | 10 | 91-93 | 92 | 122 |
| | | 320601072 | 10 | 87-94 | 93 | 123 |
| | | 320601094 | 10 | 92-96 | 94 | 124 |

1. A method of modifying maturity of a rice plant, the method comprising introducing one or more nucleotide modifications through a targeted DNA modification at a genomic locus of the rice plant, wherein
(a) the genomic locus comprises a polynucleotide involved in flowering time regulation (FTR) encoding a polypeptide comprising an amino acid sequence that is at least 90% identical to a sequence selected from the group consisting of SEQ ID NOS: 14-26, and
(b) wherein the maturity of the rice plant is modulated compared to a control rice plant not comprising the one or more introduced DNA modifications.

2. The method of claim 1, wherein the rice plant exhibits early maturity when the targeted DNA modification results in reduced expression or activity of the polypeptide comprising an amino acid sequence that is at least 90% identical to a sequence selected from the group consisting of SEQ ID NOs: 14-21.

3. The method of claim 1, wherein the maturity is modified in the absence of a substantial reduction in grain yield measured per rice plant or as a population of rice plants per unit area.

4. The method of claim 1, wherein the one or more nucleotide modifications target more than one distinct genomic loci that are involved in the flowing time regulation of the rice plant.

5. The method of claim 1, wherein the maturity of the rice plant is delayed by about 5% to about 50% compared to the control rice plant as measured by the number of days to first or 50% flowering or panicle emergence or panicle initiation.

6. (canceled)

7. (canceled)

8. The method of claim 1, wherein the stature of the rice plant is not significantly altered compared to the control rice plant as measured by a reduction in plant height.

9. The method of claim 1, wherein the targeted DNA modification of the rice plant does not substantially alter root architecture of the plant or does not significantly increase root lodging, compared to a control plant not comprising the modifications.

10. The method of claim 1, wherein the rice plant exhibits first flowering about 5 to 15 days earlier than the control plant.

11. (canceled)

12. (canceled)

13. (canceled)

14. The method of claim 1, wherein the targeted DNA modification is selected from the group consisting of insertion, deletion, single nucleotide polymorphism (SNP), and a polynucleotide modification, such that the expression of the FTR polynucleotide is reduced or affected.

15. The method of claim 1, wherein the targeted DNA modification targets the genomic locus of the FTR polynucleotide such that the one or more nucleotide modifications are present within (a) the same coding region; (b) non-coding region; (c) regulatory sequence; or (d) untranslated region, of an endogenous polynucleotide encoding a polypeptide that is involved in maturity.

16. (canceled)

17. (canceled)

18. (canceled)

19. The method of claim 1, wherein the targeted DNA modification reduces expression of the polynucleotide, reduces transcriptional activity of the polypeptide encoded by the polynucleotide, generates one or more alternative spliced variants of the polynucleotide, deletes one or more DNA binding domains, introduces a frameshift mutation in one or more exons of the polynucleotide, deletes a substantial portion of the polynucleotide, deletes a full-length open reading frame of the polynucleotide, represses an enhancer motif present within a regulatory region encoding the polynucleotide, or modifies one or more nucleotides of a regulatory element operably linked to the polynucleotide, or any combination thereof.

20. (canceled)

21. The method of claim 1, wherein the targeted DNA modification is introduced through a genome modification technique comprising a polynucleotide-guided endonuclease.

22. A rice plant exhibiting early maturity comprising a modified genomic locus involved in flowering time regulation (FTR), wherein the genomic locus comprises one or more introduced mutations compared to a control plant and wherein the FTR genomic locus encodes a polypeptide that is at least 90% identical to the amino acid sequence selected from the group consisting of SEQ ID NOS: 14-21.

23. A rice plant exhibiting delayed maturity comprising a modified genomic locus involved in flowering time regulation (FTR), wherein the genomic locus comprises one or more introduced mutations compared to a control plant and wherein the FTR genomic locus encodes a polypeptide that is at least 90% identical to the amino acid sequence selected from the group consisting of SEQ ID NOS: 22-26.

24. (canceled)

25. (canceled)

26. The rice plant of claim 22, wherein the stature of the rice plant is not significantly altered compared to the control rice plant as measured by a reduction in plant height.

27. The rice plant of claim 22, wherein the maturity modulation of the rice plant does not substantially alter root architecture of the plant or does not significantly increase root lodging, compared to a control plant not comprising the modifications.

28. The rice plant of claim 22, wherein the rice plant exhibits early maturity in a range of about 5 to 15 days as measured by first flowering timing.

29. The rice plant of claim 23, wherein the maturity of the rice plant is delayed by about 5% to about 50% compared to the control rice plant as measured by the number of days to first or 50% flowering or panicle emergence or panicle initiation.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. The rice plant of claim 23, wherein the stature of the rice plant is not significantly altered compared to the control rice plant as measured by a reduction in plant height.

45. The rice plant of claim 23, wherein the maturity modulation of the rice plant does not substantially alter root architecture of the plant or does not significantly increase root lodging, compared to a control plant not comprising the modifications.


SEQUENCE LISTING (62 sequences)

SEQ ID NO: 1 - DNA, 2540 bp, Oryza sativa
gcaagaaggcaagaaccccagttgagagtggagatggccattttgttcaggcactaggcagtgggtaggacggtgaggagtgtgtcctgcactcctgcttcccctgacgatcgatccccatgcgtgccgaagcatctgatacttttgctgtgctgttctttaatttaccctgttacttttggatatctgaagcagtagagctcgttcactcaaacaggctagcctgttttgctttctggtaagaacttaattaactagttgagatcgacatgcatggatgcgtgaatctattgttgcattttttctaatggatagttagatttgttccagctttttttttgtcaattctgttacatttgcagtgtcttcaacttctgcatcattcttctctattcttccttcactttctttcttgaactagaaaagcaaaccagagttctttttccccctcttctacataaacaaaaacagcttgttgactggctccctagagctttttgtaagttgatcatcgaagtagctagttctcttcacttatcagtcatcactgttctatgttctatctgcattttccttgattttgtacttttcctgaacgaaaggacaatccttagccatcataatgctatgatcgacttattctgaagtcatccggcctcgatcttcttttgtttcggtgagatctgtaatggtttaggaaaattatggatctctgaaaaataagaaacatctagtagtatatgaaattaagaaatgtcggaccacgtcaaatcctactggtatcatataccagataactaactttttgaatggaaccaaacacgagattgtgaatatatagcagtaagaaatacaaccccgaagggtaaaggagaaaaaggaaaagcagtttagtgtacgagctgtaggagtagctttgctcctgccaaggcccagatccttgcctcttccttcatttctgcgaggagaccaggcactgaggtctcattgtgatcgaagattctcctgtttctctccttccagatctcctttgggaacaccctgtgtgtttgcaagcgcttagcttcattttcatgcatgatatgatctctggatagaagataagtagcccccacacatgcacttcgccacctatagccagcctcgagttctcatctgtttgatccgtcgcacatgtggagcaacgaagctctagagtacgtctcctaatctctacttacttttgcagtttgcacacatattgagaaatgcttatcaatccttcgatctctcacagacaatacctacaagcgtcgtttacactttgcatgtatagctagccattgttacatacgtggagcatgtagcgatcattcgttcctagcacgtaaattaatcgccgtaattttgcccatgcagacacagcacacacagctacaaatcgactgtaattaaggtacgtatatataggtgacaatggacaaccagcagctaccctacgccggtcagccggcggccgcaggcgccggagccccggtgccgggcgtgcctggcgcgggcgggccgccggcggtgccgcaccaccacctgctccagcagcagcaggcgcagctgcaggcgttctgggcgtaccagcggcaggaggcggagcgcgcgtcggcgtcggacttcaagaaccaccagctgccgctggcgcggatcaagaagatcatgaaggcggacgaggacgtgcgcatgatctcggcggaggcgcccgtgctgttcgccaaggcgtgcgagctcttcatcctggagctcaccatccgctcgtggctgcacgccgaggagaacaagcgccgcaccctgcagcgcaacgacgtcgccgccgccatcgcgcgcaccgacgtgttcgacttcctcgtcgacatcgtgccgcgggaggaggccaaggaggagcccggcagcgcgctcgggttcgcggcgggagggcccgccggcgccgttggagcggccggccccgccgcggggctgccgtactactacccgccgatggggcagccggcgccgatgatgccggcgtggcatgttccggcgtgggacccggcgtggcagcaaggagcagcgccggatgtggaccagggcgccgccggcagcttcagcgaggaagggcagcaaggttttgcaggccatggcggtgcggcagctggcttccctcctgcacctccaagctccgaatagtgatgatccatatggttccatgcatgcatcgctgaggtgctagctagctactatagctgctcaaatcaaatgctcaatgtgtcggtaattaattaatgtggtacgtattaacttaaccgatgtacgtaatggacgctcaagctaattaagggatgtacaatttactaattaatttaatttgtaatatatagccgattaactagcaaggtgacccagtactatttgtaatttcttttcccgttatgctactaattgtggacgcacaaaccattaccggaacagaaattactactgatgaattactatatataataagcttatatagatggatgaatgttttctga

SEQ ID NO: 2 - DNA, 12519 bp, Oryza sativa
agtagcagtagcagcggagggagcggcacacgatacgcgccgcgcggatcccctcccttccctcctcttcttcctccgccttcgccgcckycgccgccgccgccgattactcgcttccgcctccgcctcaaccccgttcgtccccaaccaaccaaccgcaggtaacttcccctctggatcttccacagtatgtattcgtcgattcatttcattttcatccgggttcatcagtccaatccaagcgaatactaagctcacttgaattcggttggtgcctcgtcgtcattctgccaccacttgagagttccttcatacacacaccactcgcagtctcagcagcagcagcagcagcgcaggtaatttcactttacagtttcttctactactatcatagtcctagtcctagtactatactactataatactatgtgtctgatgcaactctctctagtccgtagcaaagctgctgttatctccctacgtacgtaagctggatcacatccttctctttttttttctctatctctattctttctcatttgtgagattgatttgcacaactgcacaagcacagatgcggatgcagcccagctagctaagctaggttagctgctgctgttggtcttggaggcaaactagtgtaatatgtgccgtaccgcctgcctttcttcatcaaccaaacccctgccaccactcaacccggccaacctcttcgagtcaggctgatgatgggaaccgctcatcacaaccaaaccgccggctctgccctcggagtcggagtcggagatgccaacgacgccgtgcctggggctgggggtgggggctacagcgacccggatggcggaccaacctccggtgtgcagccgccaccgcaggtctgctgggagcgcttcatccagaagaagactatcaaagtcttgctagttgagagcgatgactccaccaggcaggtggtcagtgccctgcttcgtcactgcatgtatgaaggtctgtctcttctttcctccttctaccttctcacttctgtctatttatttatatgtttgcctttttcacaacatattttcctattattacttactatctgactctcaaactgttaatttttctgctcctcatctctgtttaatcacacgtaccgcctctgctttacaatttgctgtagtgattttatccggaaactatcttcttaaggattgtaccaaacacatttttcaccacaaattagacgattgtcaagttcagaacaagattttgaacacctacatcacaaggacgataagcaagcaacaagatcatttggtacataaatctgttgaatactttgttttgaaacataaagtatgctgttccaaagtcttgtttgagagactgacttgaaccctgcaaaatccatgcacactttgtcatgctagccgctaaagccaaatttaaactaggcctgattaggtcaactggaaactctttctgatttttttttgttgaaaaaaggggtgcagattgtggtactgataatcatctcaaggtcctcctttttttttttactgaaatcatgtcaaggtccttgaacggacacgttaccaaatcaatttgttgaagaagggagagataactttttaatccaagattaatctccacgttgtatgagaggccaccttgaattgatggtttctaatgattttgggttttcttttttatttcaatacttgtatgaatttctagtgccaatattttggaccaatgctcttgtattccttatctttaatttggatgcaacttttccattggatacatcaaacgtgaatgaagtcatagggatagtttgttgttgttgtgttgtgttggatacctccttttggcacccagttcaccttcttcttgtaagccaatgggtaccacctagaatctccagccatctgtggacagcactatatattgcaggcttgttttcttcgaagtatttttatgccttccctgtcttcgtatcatattgatgtatgattttggactggaaaatgatttgattctatttcgatcgctagcctactgttaggtttgtcctattctgctgctttggtgtggttgcttcagctcatgaagggggttgctgcggatgggcacatcaaaaccatttctttttcattccattggtagcatcacttctggggagcgaccatcaaatgctacgtagacaaccataccttgtccttctcccttggttgttggccatgggagaattgttttcattgcctaagccagagggtccttttcatcgcatgttggaaagtccagctatgcagtagcccgaaactcctcttctgccacccaacattgcctccctacaggtgacatgtgccaaatggcgacggcagcaagactagaccacccagcttggcgataggaattggaggttgtcggcctcttcttgtatgtggggtggcagatttactgccaccaccattagagtctgtttaaaagagtatatatacttggacagcaaaaagcacatgctagattacatgaaagtaggaaaagtctaggtagctcaatgaaggttttgttacaacatggtcaagtaagggtgtagggcctgattacagggcactcccaatttgattgatgagtacttctacagtgctctctgaatactacaagtattgttaccaatagaagcgatagaaggcagtgatttatgagttctactaccactagaaaaaaaatttctgctaccagtagttgaggtggtagcataaatagatatggcccattggatgagaaaagatgacaggactagattgattttactaccagtaaaaaccaccgaataaagtttctaactattctttcctcacactattgagaagaaggcacttccattaaaaaggaaaacactttcgatatcaaggcatcaatttgtttttcattaaaaaggaagctgaatgaggggataaaaaccttcctttgggctttgcatgcgaatagtctaataaatactccctctgttttcttttatttgacacctttgactccacacacaaaactaggcaatcattgtactttctagagatgactattttactcttagagaaatttagtgttgttggtagggtatgctcacacaagaactttctagattagtgtggatgaatacatagttgtttcttataagggcaaaatgggaaaataaagattgagtttcgtgccgtgccaattatttgcaaacaactttggtcaaagttgtctaattaaaagagaactgagggagtaaagaattctagctcagcctaggaaagtagagccaacaacataagtcaatgagtccatgttgcttttgagccaactaattgagcaacaagtttggcatctactacaaaagagagaataaccatgtgagaagggtatatcatttagtaggaagggtatatcctggaatacctcgcttgacctctttcaaatgattgtcttggcttggcttcggtttagctggcacatttattaggaggaagggtttcttggagcttttcttcattgcttgctggcacatttggaaacagcgtaatgaaaagatttttcagcatattgatccttctttgatgcctggtggtctgcctttcagacatgagattctcctatataactataggatgaatggcccctacgacaaatagttgttgattggttactacttttgtagttctttggtttttgtttttgccttttatgtacatattcctataaaacttttttgttctctgacatggggtagttcttcccctaaaacaaacactcaattcaccggtctgacctgtggaccggctggttgaatcatggttcaatagcgtatatatacattgtatattctctgtagatcacaagggaagaatgacaggaatggtaggctaaagctcagagttctcagtgcacggacctgagttcaagtccccatgccacaaccttttctaggattttcattaaaaaaacagaaggcataaattgggccttgatgtggcccatttatctctgcacggttcaaccggggcagtttaatgtccaatttagcttaaactaggccggtccgcccagtccaatccagctcaaatgcatgttcggtctttgagtgcaaccgtggcgaccttttgttctgtttttttaggtcgacttgttgacccggttcagtttttttacactatgacgcctggagatacaactgctgggctaaaatgggttgagcaactgtcgagttggcctgagcaaaggaagggcccacacaactctgcccatgtttctggaaagagaatcatcgctccgattcttctctcttctaggctagtttgagcaattcccaggtcagctcataacaatgtgatatcagagctcgtcaatcctagagaagtcgtgattccgaacacgtatgcagcaacaggtcgaggatatggattttaggaaagtattccaaacacataagtgtgggcctttccttgctcggcccaacccatcggttgcttgacccatttcatcagttgacccaagaattgtatttctaggtgtatgtgggtccgctatacgtgagagtgaaatctagattttggaggcaccgaggcagggtgtgatatcaagattgtaatcctactcatgagtaatacaatgaacatatcttgattgttttaaaaaaaccgacaaggctgtgatgttcttttcacaaaaagaaagaaagaaaagaagaggccaatgttgtgctggtcttttggcctgtaatggctctttatggacgagaacattgttattaagctattgttgctggtgacttgtctagaacacactacacatacactctcctcggaaatttcattagccacaagtgttagcacctccaacctaatcccatcctgaaggacctagaacttgattttatccattatctaaaaagaaaggacctagaacttgccttcccacagaaaggaattacgagggtcttgtcatgttgctagccgatgataatatctgccaaatattcttgaagtgcaggaagttacccttgtaagcatcgaagaaacaggatcctaagtgcctcgcaagcttgtgcacaacatgacccaatggttggaatagtggcaaacaccggactaaaatttagtatagactagtccactggtttgacatatgcttgttctcagtctgactatattggggagtaaaggttttttttttcatatttcaacattagaaactttaaattgttggtctagtactcatacttggctgtgttcgcatatgtgggttgggaactcattccctccgcatggaaaacggagtggtccattagcacgtgattaattaagtattagctatttttttcaaaaatggattaatttgatttttttaagcaacttttgtatagaaacttttttcaaaaacataccgtttagcagtttgaaaagcgtgcatgtggaaaacgagggagaagggttgggaaaaggggatgccgaacacagccttaagtttgttggtcgcactgtacccaagcatgtggagctttgatttccttcagtctgtacaattttgggtgagatatgcacaacagcacaagataaatatagcgtgttctaatattttaactcacccgctaatttacccagctgccaatatttttcatgattgacgtagcctgtacaaattcgatccttattttttgtaatttgttatggactagctagggtaagggtttgaatacaaggtgctccgtgggaaccattaatggcatcagatggacctttttatactggagtttgaatgtttggggaatagacaagagacttatgcatcattttattgagaaggaaagggtctggtataaatccaaaaaatgaaaaacaaaacaacaaagaaactagaaatgtcaaatgtggtgggtggtggtgaataatgctgcttgctagcgggagatgcctgcccattggctatctctcgcaaaatgatagtgtgaccaagaaaagataaagcactctatgccttgagctgtgggatcatcactccagtccagcagcacttgatcctttttttctcattgcaggcattatattcaatttgctaacgactttaactgggatcactgatttatggtccccattctttgactaaaactcatttctgcattgtgcacttgcttgctgtttcaagtattattcatatttcttgtggatcttccattcgattatactcttgtttattcggcttctttgttaaggacagggagccaagcacactagagtaatattctgaatgcagactggcttttgtgacctttgtcggccccggttttgggtgcttcttcttagttgcaatacctcggggaagttctttccccttggggccaaggtttttttttaatgcacaagatattttatctttgttggtctttgactgagtgttattaatttatttctgtgaagtcatccctgctgaaaatggccagcaagcatggacatatctagaagatatgcaaaacagcattgatcttgttttgacagaggttgttatgcctggtgtatctggaatttctctattgagtaggatcatgaaccacaatatttgcaagaatattccagtgattagtaagtagctttcctcaactctacagtacaattcttgtacaaaatgttgcccttgtcatttatttactttctatgcttttagctgctacactgttgtggtttatgtgcagtgatgtcttcaaatgatgctatgggtacagtttttaagtgtttgtcaaagggcgctgttgacttcttagtcaagcccatacgtaagaatgaacttaagaacctatggcagcatgtgtggagacggtgccacagcgtaagttgttgttggtccagtttttcttatatattagatgttcaggcaaatccatcaagtactgattgctgcttcaccattttatggttacaatatgtgatgactctaaactaaagttgttattttcaaattcagtccagtggcagtggaagtgaaagtggcattcagacacaaaagtgtgccaaatcaaaaagtggggatgaatccaataataacagtggcagcaatgacgatgatgacgacgatggtgtaatcatgggacttaatgcaagagatggcagtgataacggcagtggcactcaagtatgaaacttgatctttttattccaacatagctttactactacctgttaacaaagctgtaattagaatgagaagaaaaaactgaagttaaaaactgaataaacctgtcagtaacaatgatttctgaaggcataaatgacatttttttgcatagctgataaatttattttagaatagtgggaaataggaagagttcaccatgtcactgtaaagtttttgaattaaccaaaccagtaaaataccatggatcatctgcatataacaatcttaatattgtacaaacacaacagatgaaacacatactgaagaaaatatagttatcgcctctcgtatagtttacatgtgtgttcatgtggcactatcgttttgcttaactaataccaaggtgccaaataaaccagcggttggttcattgcattttggagcacatatagtatttataaatcttgatagcgtgtgtgtagaatgttccactagaagttgatagcactgtgctttttgcatgttcaaggaatcaaatggattgtacaatacatgcaaccaaactttgatgtagggttttgctttgttttggcttggatgtccaacttatagtttctcaccagttggattaacttcttttccttgttctcccattgtaaacgtgaattgaacaatccttgccttttaagataaatgtggggtctatgctttatgctgccctttaaatttttgaaatgctaataagtagtttgcatgcatcgtgcttctctaaatgtcatcaagttgactaatatacagttcactatcaatcagctaataccactttattttatgtgtttcctattataaattgaacattttgtttccttaccaactagaaatcatactacagatacatattgtatagttattttcggtttattcatgaatttgtgttagaattagactatatagacattgctggttaaaaagtactcatcccctaacactgtgggaagagtacaaaactactagtgtgttaacaccattggcctcttgacattcaaaactgctacccaatgaacacattccccatcaatggtccaataatcccgaagtctaaagtccttaatcaactcgtgcataattattcacttggtaaggaaataggaaaaggatggatgcacacaaatgttttgtccatttttgctaagcctccagtatttttacttctgtgttttgagcttcataatggtttccatgtcttttcttcaattaacgcttttgtcctgtattttgatttctatttgcaccatttaatgaatcttccaaatttgggtttttcctcttgatgatttttaacttcacatctcaattgatctttaaaatatgcctaattatttaatactacctccgtcccaaaatatagcaacctaagactggataggacatatcctagtccaatgaatctggacaacccttgtccagattccttggactaggatatgttccatccaatcctaaattgctatattttgggatggagagagtatcatctaattaattacctgcaaatctgcaatgctctgcttaaacatattgtatattttctatactaatttactatacttattatcaaaatgaaacgtatattaaaaattaaatatctgctgcaatatgtgccctcaaaactagtataaacatattgtatattttcttttcaaagttacctttaatttggcgacatgtttggattgataaaatcaagtctaagcttgcaaatttggttctcctgatgctgtatgtttgattccactaaatagattctcttcaatgacctaggcaagctgtcgtagtttatcccaaatgtgttgaaaaggttgttctgggtatcaccgtacagatagattgatcctgaaattcgtatgcagagtaaacaaaatagtcttatcaacatagataatgctaaacatcatatcatcagcgcactagtgcaggactgccttgacatataatggttgcctgaaatggcaatgaaataaaaaatattttatttaaaaagcaagattcaattgtgaattagcaatagttggagaaatttagagttacaggatatggcacacgaggtgtacttgagaactgtcggtttgtagctagagatggctacgaatgccgccggggggtacctagcttatcggcggtggatgaaaactagtgtaaacttgaaattagtccacgtttcatgtggatgaaatctagtgtaaacttgaaattagtccacgtttattatgaaacgttgactacagaccctaccgagataaactgtatcttatgtgttgctgtcaagttctcttttattacagtgttgtggagtggggtaaaatttttttcctgtcagttagttatgaatcacataagattttgccactgaaatgttcagtgacatggccgatatgtcaatgtaagaaacaacttagaataaattccacattacaatattcagaaataattactgtagatcggatcactttgtgaagtgcatttaaactggttgctcatcatttttttaaggcgcagagctcatggacaaagcgcgctgttgagattgacagtccacaggctatgtctccagatcaattagctgatccacctgatagcacttgtgcacaagtgatccacctgaagtcagatatatgcagcaatagatggttaccatgtacaagcaacaaaaattccaagaaacaaaaagaaactaatggtattgtatgctcaactgattcattgtgcaactcgataaaacaaaagctccataacactgtatattaataaatttcaacatgtttttttttcagatgacttcaaggggaaggacttggaaataggttctcctagaaatttaaacacagcttatcaatcctctccgaatgagagatccatcaaaccaacagatagacggaatgaatatccactgcaaaacaattcaaaggaggcagcgatggaaaatctggaggagtcaagtgttcgagctgctgacttaattggttcgatggccaaaaacatggatgcacaacaggcagcaagagccacaaatgcccctaattgctcctccaaagtgccagaagggaaagataagaaccgtgataatattatgccatcacttgaattaagtttgaaaaggtcaagatcgactggggatggtgcaaatgcaatccaagaggaacaacggaatgttttgagacgatcagatctctcggcatttacgaggtgcaaaacataatatcagtgtcgctagtgagttaggaaaccattgttaagttgcatactaactgttacttttgttgcaaggtaccatacacctgtggcttccaatcaaggtgggacaggattcgtgggaagctgttcgccgcatgataatagctcagaggctatgaaaacggattctgcttacaacatgaagtcaaactcagatgctgcaccaataaaacaaggttctaatggtagtagcaataacaatgacatgggttccactacaaagaacgttgtgacaaagcctagtacaaataaggagagagtaatgtcaccctcagctgttaaggctaatggacacacatcagcatttcatcctgcacagcactggacgtctccagctaatacaacaggaaaagaaaagactgatgaagtggctaacaatgcagcaaagagggctcagcctggtgaagtacagagcaacctcgtacaacaccctcgcccaatacttcattatgttaatttcgatgtgtcacgtgagaatggtggatccggggcccctcaatgtggttcatccaatgtatttgatcctcctgtcgaaggtcatgctgccaactatggtgtcaatggaagcaactcaggcagtaacaatggaagcaatgggcagaatgggagtacgactgctgtaaatgctgaacggccaaatatggagatcgctaatggcaccatcaacaaaagtggacctggaggtggcaatggaagtggaagcggcagtggcaatgacatgtatctgaaacgcttcactcaacgagagcatagagtggctgcagtgatcaagtttagacagaaaaggaaagagcgcaacttcggaaaaaaggtagcctgttttcaattgcatgttttctgttcctttggttttagcattcctgtttaactcgtctaaattagctaaagaacatgttactggaagtagttgtcaaaagcatattactggaagtttctcccaaacgactagctaaatgggatcgggcatgaatataatttgtttatatactagtattatctgatttctaaaaggaatctcacaacataatcttccaaaaagttgcacgtttttggtcccatagccgtgttgctgaaatttcttgccaatgacatttcttggatttttctcataatttaatggtagctacttagagcggcaattcaattttactcttgaaacactgtcctttacttttcggcgcgggtgagtacaatttggataggaggctctttttatatatagagaaacatacatacatacatacatatatatatatatatatatttatatatatatgatgtgcttgattaagctttggataggaccggtttctttctgatggctgtctgccattttcaggtgcggtaccagagcagaaagaggctggccgagcagcggccaagggtccgcggacagttcgtgcggcaagctgtgcaagaccaacaacagcagggtggtgggcgcgaagcggcagcggacagatgacctacctacctacctacgcgatggctttggactccaaacagctaattaacagttagtagacaacagataatgattcttcttccttggccgatcgatcaacaacatcccatgcatccggcatcccaccaccattgattccatcatatttagagtctggaataaataaggaactcctatcctatttatcccctatctatatatgaagatatgataatggtgatctgcgttactactagtagaagaatatggtgtggctgactccacttcaggtggacctataatactactccattagtatgtgcctgtggaggcaagctcgaacgtactactccatatttaagcatgtcatgtactgctactatgagacgagagtgctctgccctgtagggacagcactattgtcaatgtcatgtgtttgttgggtcactggtcttcttagatttgcgtccgtgtctggcagcagcactccactgtagttggcccacgcatgttgttgaaatgagccacatgccttgccttgagatagaacttgctgtcactgtttctccttaatctaaatatactggagtggagtattttattatctatgatctgtaatcaggtgattgacaaggctcgtcaaa

SEQ ID NO: 3 - DNA, 2784 bp, Oryza sativa
atggggatggccaatgaggagtcgccaaattatcaggtgaaaaaaggcggccggattcctccacgtaaggaccaaatccatccacagatcgccccgctctcctcgatcgatcataatatgatctcgcaatggcccccctacctttccctcatccccaacttgccctgtcttcttcttcttcttcttcttgtacctatattattacaagtcatcgatctcgctgatcgatcagtgatcacaagcatttcacaaccctagctagctgagctgatcgagctcaagtgacctcacctgctatagctaacttactagctagctctagctagttgttgtttgtagctcgatcgagtttgatttatccgttcatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcggacggcccgacctggacgccgggctcggcctcggcggcggcagcggccggcacgccgaggccgcggccagcgccaccatcgtgagtatcaatccaataatcctgatccggccggcatgatcggctcgatcgagccgtgtcgattattaatttccatcttatatattattaattgatgaattcttgattgattcatcgatcctcctcgtcttttcttggcttccttgtttttgttatttagtcaaaaacaactcttcatttctgctgcctatatgccgtacaacttcaaactatcaaaggtcaaataatcgatcaatatataccaagtttgaattaatttggagcttaattaattaattactggcttgcagcagctggtttatagtattgtttctagctatatatgtgagggccgtgtgtgggatgtgatttgcatctttcgatggcgacttaattaattcgatgatatatttcattgcatatgcatacggatccagcctctgtctatactgtacgattccacatacgtatatgtacggttaagtcagtatatatacttttagatagtcgcgtgtgcttttcgagttcggtagctatattttagattgtaaaaacaagtcagaggctaattttataatctagaaatacttatttccccgtatataaccgtatgttaaatattgatggtgtaatctacttataagtcaggaaacatcattgcttgctttctggcgctttcttctacatatcagtagaggaaaatggaaaaaaaaagatgaattttgatgttgtagtttgctatattcagcatatatgccatcagttatacatatgcagatcttgctaaaaccaaaataaaaatagaactgtaaggagatattgtgcttctcggtctatttacttacagtttgttgagaagtaatacgagcaagcaaatgtatatatatatttctttagaactgcaaggagatgcatatacatgtgtgattcaaacacacgtactgcacattcaaactataaaaacaacttgattgccgtagaagttaaaagggagacatatccatgggtttcggattctaaatcaatctatgtgtaaatgaaactttagtatagtaggaaataggttttcaaaaaaaaaagtatagtaggaaatagtatgtgtatatgcctttttaacccttaattacaagttgtaataattcagtgttaacaaagtcacggactcacagagtgtgcccttacacaatttcagactaatttgtaaatgcatcgatcgtcacattttatgtggttcaattatctgacacagttaattaatggtggccgatcgatgtatgctcctctagctttccagctatatgcgtatgtaataaatgaataaaacgtgtaggatgaaatgtgaatacgcatcattgcaattaatttgattaatgctagtaaaaaatctgcaaatttgtctttttgaaattaaaatatgccttataaaattaatggacccaggcccctaggccaaaatatattggggcacaaaatcatgtccatatatacattcttatttgaaagtagactctgaaacaaaatatgcccatataaatcaagggaggttacaactaactgcatttgcttatgcgtacatctggattgtaacttctacgttttgtacatacgatgattaattgtattcgagcttcttaattgtacatctattaactaactagttttgcagatgtcatattgtgggagcacgttcactgacgcagcgagctcgatgcccaaggagatggtggccgccatggccgatgttggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaagctgatcaggaagctgtcgcaccgccatccacctatgtcgatcctagtaggcttgagcttggacaatggttcagatag

SEQ ID NO: 4 - DNA, 3120 bp, Oryza sativa
attcccctttcccctcttcctctcttcactcacctcttgcctgtgcaagtaaactcagcaccactacccctaccatcaccaccaccaccactactccatctaccatctccagtgcctctctctctagcttcttccttctttcatggcaggaaaggctggggtgatgatgcccatgagcccatccattttgacccatgccatcctggtcctatatacatactccccgtaggctcacactcccaatctatacttgctgctgcttctgcttgcgattgcgagctctctcctctctctctgttttttttttttttggcttgcaaagcttttgctttgttttaggtgcttcttttccactcctttactgttcccccacttttccttgctttgtcacgcacacgaaaagctcctgcctttaccttatctccgctcgccttccctcacccccgtcgtagtgggatttggtggctgctcgcctctcgcgtttcttttgctggattggggcgttcttgggagctcgggaattcgggtggtttggttgggtttcgtggagaattgcggcagatctttgagatgaactgcgggccgcccgaccagttgccgccggcgacggcgccgtcgtgcttcctcaacctcaactgggaccagtccatggacgcggcggcgggagggcacctcgacccggcgctcagctccatggtgtcctcgccggcgtccaactcgacgggcgctctccacgggatctcgccgcagccgcactacggtggcgggacgccgctcagctcgccccccaagctcaacctgtccatgatggggcagttccaccactacgccgcgccgccgcaggtgggcggcggcggaggcggaggcgggggcctgccaatcctcgagaacctgatgcccatgggccacctcgaccagttcctcgccgaccctggcttcgccgagcgcgccgcgaggctctccggcttcgacgcccgcggcggcggcggcggaggaggctacggcggcgccggcccggcgcaattcggcctccctgacgccggcgcggccggcgcatcgaaggagatggagctcgggaacacccgggacgagtcgtcggtgtctgatccggcgcccggcggcgccgagattccgcccaagggggcttccgacggcaatgcacggaagcggaaggcctccgggaagggcaaaggcaaggacagccccatgtccacctccgccgccaaggtacgcgccgaattccgctcgccatggccgcgccgcaccattcttggaccgaaagatttcatctctcgagcgttctgcaggaggattccagcggcaagcgttgcaaatcgacggaggagagcaatgcggccgccgaggagaattccggcaaggggaaggccgcgcagagcaacagcgagaatggcggcggcaagaagcaagggaaggacagctcgtcgaagccccccgagccgcccaaggactacatccatgtccgcgcccggcgcggcgaggcgacggacagccacagcctcgccgagagggtacacacacaaaattcttgcagctctagcttccattgctcttctgatcatcatcttccctgtgcattaacatgctgtgtgtatctcctcttgttcgatcaattcaggtgagaagagagaagataagccagaggatgaagctgctgcaggatctcgtgccgggttgtaacaaggtaactgaagaaaagcactcatcatttgcatcatcgcccacctgtttgttacttgagttgctcggtgtgctcagattggaaactgggaaatgcttacttgtttcaggtggttggaaaggctgtcatgctcgatgaaatcataaactatgtgcagtcgttgcaacggcaagtcgaggtatttgtccagtgtcaaattcacagatgcagaatgattgtgaaactgtagcttttgccgatttagagcgcagcaagcgcgggcgtgcgccatctgaacctttgcctttcacatccattttgcagtttttgtccatgaagttggccactgtgaatccccagctggacttcaacaatttgcctaacctccttgctaaagatgtgagggcaagcttatgtgatctcattactacatttgtttcctctcttattgtggcaatcatacagttcccagcttacctcgtgtttccgattttctccctgatcagatgcaccagtcatgcagcccgttacagagctcacattttccactagagacctcaggtgcgccgctgccctacattaaccagcctcagcaagggaaccctctcggttgtggcctgacgaacggcatggacaaccagggttctatgcacccattggacccggcattttgccggccaatgggctcgcaccatcctttcctcaatggggttagcgatgcggcttctcaggtaaaaatgcacaccaatgtctcgatctcacaaaatgttcgaatgaatcgatgctgaatatgaggaatgaatgctttacaggttggtgctttctggcaagatgatctccaaagtgttgttcagatggatatggggcaaagtcaggagatcgccacctcttccaatagctacaatggtaggcaagtttcctcatcctattacctgatgccatagttaacgtcaccttaactgaattctaatgtgtaaactgctatggatcaggatcgttgcaaacagtccacatgaaaatggagctttgacatgaccaaataaaggttcgatccaaagctctgacgcccattgtacatacagagaagtcagcttgccgcggcagcactctgagattcaaccctaacaatgttgggccctagcagtgttttagcttctaatttcccatcttggtgtaatgattctttgaacaaaaatgaaacgcaccatgtacggtagtcgggtgtagtatatatatgtatgctacaagatcgtacataagtcgggtgcagtatatgatgcttcattcagacccttttaccctctcaagcagagagggacgacgtgtaatatcattatttgtattctcattatatttgtagctgatatatatctttaagatccttcttcctgtccatggatcgatctccttaagctgaaattatcccctttttttcccctctg

SEQ ID NO: 5 - DNA, 973 bp, Oryza sativa
gccattgctgcagtagcaagaaggagagcgagagagcaaagcccaagttggtagccatgacggagctgttcgacaccgccgtgaccagcctcctccacctaccggaggtgctcgaccgcctcggcgccgccgccggggaccgccgctcggcgggcgaccacgcgcaccacgccgcgcatggacacggccagcaccgcatcagtggaatcggtagtggcgcgccagtggacatcatggagacccccggcgagtacgcgttcgtgctcgacgtccctggcctctccaagtccgacatccaggtgcgtacaaaatccgagccctgaatttcctcgattcgcgttcgaatcagcgagttagtttgggcgcgattctttcttgtctgcgcaggtgacgctggaggaggacagggtgctggtgatgaagagcagcaatggcgccgggaacgggaagcggaagcgggaggaggaggaaggggagtgcaagtacatccgcctcgagcgccgcgcgtcgccgagggcgttcgcgcgcaagttccgcctcccggaggacgcggacaccggcggcatctcggcgcgatgcgagaacggggtgctcacggtcaccgtcaagaagcggccgccgccggagaagaagaccaagtccgtccaggtcaccatcgcctgaatcatagcgtagtgctcgggtaatggcagcgtggcggtagtagaagctggaaatggttgccgagaatggttggagcagtagaatgtgtttgtgctctgttcgtgtttcggtagtactagttgtcgctgatctgctatgtataaacgctggtgtgaccgtgaagatgaagactacggaagtagctgttcttttgtgtaggatcaagcaaattatgtcaaatctcgttcgtgaacgattccaaatcgatgcattatgttatgatctctgtcttatatgaatcctacagctacaattcttcgtttcctcaatc

SEQ ID NO: 6 - DNA, 3450 bp, Oryza sativa
gaaagaagagagagagaaagagagagagagagaggccgcttctctagtctagaccaaaggaaggaaaccaaccgagaggcgaggcgaggcgaggagaggagaggagaggaggaggaggagggaggagaagatgagagagatcatcagcatccacatcggccaggccgggatccaggtcggcaacgcgtgctgggagctctactgcctcgagcacggcatcgagcccgatggcaccatgcccaggtgacgatgaatcccccctccccctcccccaacctcttcttctttgatccgtagatgcattcctgttcctgatctcgggaggcggccccgcgttccccggcttcgtttgatctggtagccccggttgagttgcgtccgaaatgccagatttgtttcgccgtctgtttttttttgtgtgtatgtgttttttttggggggaaattgtttgcttcggcggagcgaacgatgggtgcagccttccgcttgtacttctcgtttggactctgcggattggttttggccgaaccttcttggtggttggtgcatctccttgccggttggtgaggggattgcaagggacaggacgaacaccgtgcgtctcggaggaagggaattttagccgtagtttttacgctcttttttttaccgtttggaattatatggaggaatgcgtttgattttctccggagtagactgtagatttgcgtgctggttgcatgtttacaagttggcaattgggagtttccagagttctttacggttctagatctgcgaatttacttgtggggtgatgcttagtaaataatgtcacatggaagctctgagtgtcagactgagcacgtagatcgtcctagattggagagtttaatctgggttactattaaaaccggcatcctccactatcgaggaggatttgttttgttggttttactttgcttggtgtaagaattgagtagattattgaatcataaacaattgtgctaatgctgccttgcctgagaattagattccattagattatcatggatgtagcccgctttttgtaggcagctctggtagactgtaatttatttggtactgcataggctaatgattagaattatcagtcagttttgatttctaaacattgtgctgtttgctcttggtgcagtgatacaacggttggcgtcgcacatgatgcgttcaacactttcttcagcgagacaggcgctggcaagcatgtgcccagggccatctttgtcgacctggagcccactgtcatcgacgaggtgcgcactgggtcgtaccgtcagctcttccaccctgagcagctcatctctgggaaggaggatgccgctaacaactttgcccgtggccattacactggtaattcagcttagagttcatacaagtgttattctattgcaactgtgcatgtgtcttgttctcattgtttgtggaatgcagttggaaaggagatcgtagatctatgcctggaccgtgtgcgcaagttggcagacaactgcactgggctgcagggattcttggtgttcaatgctgttggtggtggaactggatcaggacttggttctcttctgttggagcgtctctctgttgattatggaaagaagtccaagcttggcttcacaatttacccttccccccaggtataacttaatctgatccatgttagttgtactctgagggaaaaaattaaataaaatgctctataccggaaacttgaaattttcaggctggtgtagaattaatttatttggacacctgcttagatatgggccatgttatcagccacttctttttcttgttgttgtgagttgtgcatggcaatatatttattattggatctgccaaccattctctaattagtatgtggactaataaactgctggcctggctgcctggctggtggggatctacctatatggctatacaatatccctatcttatgtagactcttcagtgaacttttatggactattagtcttcttcgcgaagtctgtaataccatgcattgtatgagccagcattatcctagtgtactgtaccaactttccatgtgatgtgggcctttcagcttgttcatgtgatgagtgggtttgctttgtcttagctgttgtggggacaatttgggtagagttgttgtacaattatttaaagttcatcatattattatcttctcgtcttatgccactctggtttgcaggtctcaacagctgttgtagaaccatacaacagtgtcctctccacccactccttgcttgagcacactgatgtggcagttctcctagacaatgaggctatctatgacatatgccggagatcccttgacatcgagaggccaacctacaccaacttgaacaggctcatctcacagatcatatcttcactcaccacctctctgaggtttgatggcgctatcaatgtggatgtcactgagttccagaccaaccttgtcccataccctcgcatccatttcatgctttcatcctatgcccctgttatctctgctgagaaggcttaccatgagcagctttctgtgcctgaaatcaccaatgctgtttttgagccctcaagcatgatggctaagtgtgaccctaggcacgggaaatacatggcttgctgcttgatgtaccgtggtgatgttgttcccaaggacgtcaatgccgcagttgcaaccatcaagacgaagagaactgtccagtttgttgactggtaagctgtcttcatcactattttagttgcgcttattttaatcacaggcaacctatccacttgtttatcaacacctgattggctaaccaactgtttaatgcttctcttcctccacaaaggtgccctactggattcaagtgtggcatcaactaccagccaccctctgttgtccctggtggtgacctggctaaggttcagcgtgcagtgtgcatgatcagcaacaacactgctgttgctgaggttttctcgcgcatcgaccacaagttcgacttgatgtatgctaagcgtgcgtttgtgcactggtacgttggtgagggaatggaagaaggtgaattctcagaagcccgtgaggacttggctgcccttgagaaggactatgaggaagtcggtgccgagggtgcagacgacgagaacgacgatggagaagactattagtagctggttaataagtagttctctggttaatgattggtttgtcagtatactccggttccattgcattgcatattcctgttatgtgtgtttactttttgtactgtgctaaactgttgttagcccctcgttggccatgattgttcatatcttcccattttgctgtgcattgcttgatactcccccccaagcttggtacatatgccattctgagctgaattgcaaaactgtatgctctctatgcatggtccaatggagctatggatgctttcttctattaatctctctgcgcgtttactcctt

SEQ ID NO: 7 - DNA, 1448 bp, Oryza sativa
cattcgagaaatccctcacaacccacaacattttcaaacaacgcaaagcagtagcagcagcgagaagcaagcaagaagcgatggggatggggatgaggagggagagggacgcggaggcggagctgaacctgccgccggggttcaggttccaccccacggacgacgagctggtggagcactacctgtgcaggaaggcggcggggcagcgcctgccggtgccgatcatcgccgaggtggatctctacaagttcgacccgtgggatctgcccgagcgcgcgctgttcggcgccagggagtggtacttcttcaccccgcgggatcgcaagtatcccaatgggtcacgccccaaccgcgccgccggcaacgggtactggaaggccaccggcgccgacaagcccgtcgcgccgcgggggcgcacgcttgggatcaagaaggcgctcgtgttctacgccggcaaggcgccgcgaggggtcaagactgattggatcatgcatgagtaccggctcgccgatgctggccgcgccgccgcgggcgccaggaagggatctctcagggtaagcttagctcagattcatccgaattactagcaattatccttgcttcgatcgaagattattcggtgggagtgatgatcgataattggatcgtggtctgatctgatctggtgtgaattgtttgtgcagttggatgattgggtgctgtgtcggctgtacaacaagaagaacgagtgggagaagatgcagcaggggaaggaggtgaaggaggaggcgtccgacatggttacgtcgcagtcgcactcgcacacccactcgtggggcgagacgcgcacgccggagtcggagatcgtggacaacgaccccttcccggagctggactcgttcccggcgttccagcctgcgccgccgccggcgacggcgatgatggtgcccaagaaagaatcgatggacgacgccaccgcggccgccgccgccgccaccaccatccccaggaacaacagcagcctgttcgtggacctgagctacgacgatatccagggcatgtacagcggcctcgacatgctgccgccgggcgacgacttctactcgtcgctcttcgcgtcgccgcgggtgaaggggacgacgccacgcgccggcgccggcatgggcatggtcccgttctgaggtgacggcgacgcgatcgaacaggtggtgatcgatgctgcaacgtgtgtaaatatacagcgccggctgggtcaagagatggctcgggtgacgcgcgcgcggcgtgtcctggcgttggcgccggggcattctttagtttttcatcttttcatcatctcagatggtagatacaaaacagtgtatgtatgtagctctgtttctctctatagaaccccaacaaattttgttgttgatgttgtttatcttcatatgctttgatcttgaaatcgtctaccttactactgccga

SEQ ID NO: 8 - DNA, 1071 bp, Oryza sativa
agagaacaagaccctagctacctgttcagtgttcaattcgcacggaaagccatggcctcccgcgacgccgccaccttccaggtctaccggcccatggcaatgccaacgccggcggcgctgccaccgtcgtcccagcagatcacaatgccgttcacggcggcgcccgtggacgccgtgctcccagctccccgaaaggcggcggcgacgcagggcggcaaggaccggcacagcaaggtgaacgggcgcggccggcgcgtgcgcatgccgatcgtctgcgcggcgcgcgtgttccagctcacccgcgagctggggctcaagtccgacggccagaccatcgagtggctgctccggcaggccgagccgtctatcctcgccgccaccggctccggcaccaccccggccgtcttctcctgctcgtccgccccctccaccgcctcctcctccttcctcctcggcaagcgcccgcgccaggaggaccacgaggcgccgacgttctgggaggcgctgcagcaacagccgcggcccgccgtgtcgtcgtggggtgccctcgtctcgccgtcgcaggaggcgcaggcgtacgcgtcgtcggtggcgcaggtccaccacctcaacctactctccgcgctctccggcgccgccacgagacggccagcacaggaggagtcccggtgatcgcgcgccgcagctatgcagctcaatttgcgacgtgattgcatgcgcgcgcggggttgatttgtacttggagctagtcttgctttgtcttgtgtgtatggcttgccgtacttaatttcccatttccgctggtgtttccatgggtgaattttgtgtgaatttgcttgattttaggccggagaatactagtaagtagctggtgaagaatcaagaatgtgatcagatagatcatcagaggcagcaattgtagataccaagtcttttggattcttgggtttgtgaaattgcgattaattgtctgtcctttctagcagtgtgtgtgtgtgccgttgccatggttctgtcatctgtgacatgacgatggtgtttggttcagtaatcagtacacaaccttttagc

SEQ ID NO: 9 - DNA, 10408 bp, Oryza sativa
atttccaattccagacaaggaaaaaaataaaaaaaaaaatccctcctttttttttttctcctcctcctcctcgcgagagaggaaagaagaagaagaggaagaaaccctcgtcgcctcctcctcctcctccgccatggtggagactcgccggagctccgccgccgcggcgtccaagcgctcctccccgtccccttcctcctcctccgcccctcccccgaagcgccccaaggtgagatgagagagatactttctttttctttcttgatttgattcctcttcttgatttgatatttttccaatcggggtggggttttaatccgatctgaatcaggcggaggcggcgcccgcgtcgccgacggcgtcggtgcccgggagaatcgaggaggattcggcggcgaccaagagcgccggctcgggcgaggacgccgcggccaagagaggtgagggggggatttccttctttttttttggggtgcgcgcgcgtgggtttgttggatcgagctgttagggttctgatgctttgttgcagatcaaggaggggataaggcggctgtcgctgtggtggagagctcgcgcaagaagaaggagcagcagcagcaacagcagcagcagcagcagcagcaggcgacgccttgggcgaagctgctttcgcagtcctcacaggtacatgggaaaaaaaatctatggggtgtaattaggagttcttgatggtatgggtattgcagggttggaagttaaaatttggggcttttggatgctgaactctgtaaatattctatcttgaggatgcctagggtagaatttagtgtacatgtggtgttctgtaggagcttttggatgctgttctttgtaaaggtttaatcttgaggatgtttagaatggaagttagtatgggtgtggtgatttgcagcttgattgattagtagttttgttatttacttgtttagagctttgcagcaataagacttggcagtctgtcctgttggctgttgcccaccctgaattagttgtttgattggttactgcattgaatttgtaatatgttatctgtttgtgttgttttacatattctagtgattaccagtactgtagtagcaattcttggcatatggtcttcggaaccttcacttcgtaagaacgtaggaaactgggataatcagagaacatattagagctggttaaagttttatcttgagaaactattttgcaagtacattaggttgcttaagttttgctgttgtaaggcagaggctggcattgctgtcatggtcaaatctgtgaagaacagtgaagaactgttcagatactaaccgaggctgggctaacctcaatgacagaacacatgcatattgaacaagaagataaacaaataaacaaataaagaaaaatatattattttccaagtgagttaaaacacatgcttacttcaaactccttagtgaatcgagtagtggattgaattgctcttctctttcctttaacctggtttgccatgtatggatagactaatggttaagtgcagcaacttctcttatgctttgtaacttttcacatgcatgggcaatctatgtagttgacacaggtgacaggacatgcttgtcacaaaaagtttacttaagtcgactaggcaaatctgatgtttaggtttgattgtttggagcttccacgttgatagatttgatgtactagtattagcaattccaaattaatggcaacttacacaaatgctctctgaactagagaaagccattaccttcttgatgtacgcatctccctgtttttctcatgcaatgatacggtcaaggctagggtttaatccaattgatggtaccgacatttcacatttagagatgctagtagcaacaattcaatcctttaaccgtctatattggttgtctcctttcattctttttcttgcagatttgttgactctaaattccttctactattgtaatgcgcaataatgcatggattatattgtcactcaatgctgatactaaatatgtgtgcactgcagagtcctcaccttcctatcagtgttcctcagttttctgttggtcagaataaaagctgtaatttgtggctgaaagaccaacctgttagcaaaattctttgcaggttgcggcaactcgaggcatgtcttttttttttctctggtgccctttttctttgtgcaatgtgcagcaaaattggcagcacttgcttataatgttttgttgattttttatgttctttcagcaaggtacatgtgagctagaagttctaggcaagaagggtacggtccaattaaacggaaggtccataactgctggcacaaaagttccccttaaaggaggggatgaagttgttttcagtccatgcggcaagcatgcctacgtatcctaatcttcttctttattcatgcaatcatacagttcctttcatcaattttcctttctttattagtttagatgtgcacagtctggaaatagctctatctatatttttgttttaccttctgctttccttcaccttcctacagatttttcagcatccattgaatgacaaaattcctaaaatggtgccgccatctcctgtcacccttttagaacctccagttgctggtgtcaagcgtctccggatggagaacagaacaggagacacttcagctgttgcagggacagagctattagcatccgtatctgatcagttgaaggacctatcagctgcatcacctgcatcagctggagagaacaatcagagattagtacgacctatggcatcatccgcttctgataaatcaaaaggaaatggcatcatccctgacaaggaatgtgagaacggggaaaatgctaatgaagttaattctaatgttgaagattctccattagatgtggctgcagcccccgtagtatctcctgatgctgtcccgaacgacatcagtcaacacaatggctttggatctgatgctcatcttggtgctgaaatcggtaagattgcaacttataagataaggccagttctgaggatgattgcaggaacaactatatcagagttcgatttgactggtgatctttttaaagctcttgaagatcagagggatctcatcaggcatcttaactcttcagccagtttacctccaagcagatgccaagcctttaaggatgggatgaaacaaggaattattagtccaaatgatattgatgttacatttgaaaattttccttattacctcaggtaatatactttgggttaatcttccttcctatattctttaacttttattctgtttattttgtgttcttaatatgcttcatatctgcgatgttacattgttaaaaaaaaactaaaatagcattcattagttattcttaattcgctatattctgtatgattagttcataccaatatagcattcattagataataggcacaacacggtcgataaagtagtttaaatgtagcatgatattcattgctctctcacatgttcatattcttacagacttccactgacgtaactttaattgtagcatgatatctgttctctcacatgtttgtgttcttgtattcatgtgttgttcatcaactccatttcatgcagcgataacacaaagaatgtgctcttatcatgtgcattcatacacttggaaaagaaggaattcatcaaacaattctcagaaatttcatcaataaaccaacggattctattatctggtccagcaggtaccttttatgatctcattgcatatttattgtcttcggagttaaattgaggtccttgaaggtaaggctgtgtgcatccttttttgctgattcttgtaggcatctgatttttctaatgcaaacatgcatcaggttcggagatttatcaggaaacattgataaaggcacttgcaaaacatttcggtgctaggctacttgttgttgattcgcttttgttgcctggggtaagttatccatgtaacttgtgcttaaaactacttggtgtcaattattattattgagagcaaaattctctttaggcgccttcaaaggacccagagtcgcagaaagatgctgcgaagtctgataagtcaggggataaggctggtagtgagaagttggcaattcttcataagaaccgctcttccttggcagacgctatgcacttcaggaggccagctgtacaaccttctagtgtgcatgctgatatcgtgggtacatcgactctgcattctgcatctttgcccaaacaggaatcatcaacagctacatccaagagctatacttttagagaaggttttctactatgatattatctcttgtttatcgatagatcattttcaactatgctatacttacccgaacatgactgcactgccattgtaggggatagggtaagatatgtgggtccagctcagcaatcgtcattgtcgcaaaggtatcttactgattgtcatgctctgatatttaaaccaaagtttattgcttgattcttccttgtctttccatcgaaacctcattgatgctatgaacattgtggatgtgccattaggttggttgtttctttggatggtttgggagaaagatattgtctgtcccctacatttaacacacaaaataattttgatgaaattcacgaccacattcataaataaacaaatacaaataagaatgttggtatgctagaatacaaatacgaacataattttacaacaaatgcaaatacaaatatcacccactcaatttggttgtataaaaatatctatttcccaacctagagtaacgaagtgtggaaataaaagaaacatgtgtttttaaatacccaactagttagacagaactcaacaaatgcatgtcgcctttgatcaaattcaaattcaaacatgaatagttttggatgatataaaatactgaatagaaaatctatttagtttttctggcacatacaattttggataggggaagtattttttaaaaatcctgactgtattcaagttcttagagtaattccactgaaaatacggttttttagctacttttttcggtgtttgacatccctagttggggttgtctcacaacattttaggccgcaggtccacctgttcttgactaaatcatttgtatttctctctagcacttcgtttacaactttacatgcaatatttaatagtgaatataatttgttatatttaatcttggagtatcgtagccaacaacctagaatttactttctacttatgctacattgctcctattgtgttacatcagagttagcttgacacgctttttgataaacctatactccctccgttttttaatagatggcgccgttgacttttgaacacatgtttgaccattcatattattaaaaaaaatgcaaatgtataagatataaatcatgcttaaagtactttgactgataaaacaactcacaacaaactaaattataattaagtaatttttttgaataagatgaatggtcaagtgtgacaaaaagtcaacgacgtcatatattaaaaaacggaggtagtagttttgtagccataacatgctttcaatttattttctttataaatcatttgaatatttgattgacctttcaggggacccagttatggttatcgaggcagagtgatgcttgcttttgaagaaaatgggtcttccaaaattggagttcgatttgacaagcagatccctgatggtaatgatcttggtggtttgtgcgaggaagatcatggtttcttctgctctggttagctcaaacttttgaatcccaaaaattatgttagttcttgcaggttctgataaattcttgtttctgcagctgacttacttcgccctgacttttctggtggtgaagaggttgaaaggctagcaatggccgagttaattgaagtacagcctcttatctaaaactcagagttatccgaatataaatttgtattctttctattgtttcataattgtatggattgtctgtcaggtcatctcagaggaacacaaagctggccctatgatagtgttacttaaggatgtagagaaatcattcactggaattacagagtcactttcatctttgaggaataagcttgaagcgcttccatctggtgtgcttattataggatctcacactcaaatggacagccgcaaagagaaggtacatatattatatctcagagattatgcaaatttgttctatgagggcttgtttatggcaatgtggcattgcatagcttgtcttttttttttcatttgatttgaactgttgtgaaaactaactttggtggcattgggtattcagttctaattggttggttctgtctctttgacacttgggtgttcgtttgcatgcaatctttgttttaatttttaatccttatcatttatcgcttgttaagtatactataaaatgtatcctgatgttgtgctatggtgtctgttgtaggcccacccaggtggatttctttttacaaagtttgctagtagcagccagacgctatttgaccttttcccggtaacaattttcattcttattggtcacaatgttctttctatcagtgcttataaatgcactatatgaacccatggttctgatataactatctgatgcattgttatatctaaaatgcaggatagttttggtagtcggttgcatgaaaggaacaaagaaagtccgaaggcaatgaaacatctaaataaacttttccctaacaaaatttcgatccaacttccacaggtgctaatatcccttgttatcttgttcaagtattatgctcaagtatattatctaatccaagcaactgaatttgcaggatgaaacattactcacagattggaagcaacaactggatcgtgacgttgaaacccttaaagctaaatccaatgttggtagcattcgcacggtaagctgtgcagataacacaatttcacatatcatgcttggcactttgggttttgaaccgtgtattctgtatattcctcttgaaactgaaatttcttgtgtgcgatgcctagcctagacagctagaccctaagcagtgttttgttttaaacgacactttgctcttttcccctcaaatgatcaatcatgcatgcataaaggtgtgcgatgcctagacagctagaccctaagcagtgttttgttttaaacgacactttgctcttttcccctcaaatgatcaatcatgcatgcataaaggcacctttctataattctgtagttactcctacatgttttattttctctcgctgtcaattatgatgtatatatttaccctcttaaatgacttctattccactgcccgaatggcctgtatttgattctcctatgcttatagttgttgaatgcttgctagctatttatgtgaatttcgtttgctttgtgttgcagtttctgagccgcaatggaattgaatgcagtgaccttgaagaattattcatcaaagatcaatcactgaccaacgaaagtgagcattttcttgttgtcttagcattttatccatggaattctgtgtgttaaattgtgttgctccaaaatttcttagatgtggacaagatagttggctatgctgttagttatcaccttaagcacaataaagtcgagatctccaaggatggtaaacttgtattggcgagcgaaaggtagttatactgttgctagtttatttttcctccatttccttttaatgaatcttctataatgctaaattaattcttctgtatcacagccttaagcatgggctcaacatgctgcagaatatgcaaagtgataacaagagctcaaaaaaatcactgaaggttactctccatctcatcatattgcaaacatattatcatactcacacagttggaatctcacagtgctttactcttaaattactttttccttcttaggatgttgtcacagagaacgaatttgagaaaaggcttctggcagatgttataccacctaatgacattggagtcacctttgatgatattggagccttggaaaatgtaaaagacacattgaaggagctggtgatgcttcctctgcaaaggcctgaattgttctgcaaaggacagctaacgaaggtagagacgttatgatgaattatccatgtctcatatgcgggtgtaagattgttctatctcaattataggttgcgcacttgattagagtgttttgtcaagtaataaggcccatgatgtgattaggtttctgttggctagtaatgaacccatgatgttttctcacctaacagtagtgaaatctataataagttattttcctctgttctccaaagttctgttctaacacattatgccaattcggcagcgttacttgtcttaggttttcttgttcaattttttaatggtgaatgggtaaatttaagttaattagacatgtgggaaatttggttgtcagtacttcctccatcccaatctagtactaggttgctatattttgggacggatggagtattatatgttggctacctaattttctctatttaagtcctatcttaaaaattgcctctggtaaatctgttacttgcagccttgcaagggaatattgctgtttggacctcctggtactgggaaaaccatgcttgctaaggcagtagcaactgaagcaggtgctaacttcattaacatatcaatgtcaagcattacatcgaaggtatgatggcatccaaattttgcattaatgaggtgtacagtgtactatgtgattctcaaagctaaatgctgttttttctactttcagtggtttggcgaaggagagaaatatgtaaaagctgttttctcattagcaagtaaaatagctccaagcgttatattcattgatgaggtatgcttgagcatgtgggttggttttattgtcattagtgatttatttggtcacggtatgatccatacaaaatgcaacatttggcaggttgatagtatgctaggaaggagagagaatccaggggagcatgaagcgatgcgcaagatgaaaaacgaatttatggtgaattgggatggattacgcactaaagataaagaacgtgtactggttcttggtgccacaaataggcctttcgaccttgatgaggctgtcattaggaggttcccgcgcaggtgacagctataaccttgtgttggggttgggataatgaaaaattgctgtttgttgatttctaaccatttggtcgttaattactactgtaggttaatggttaacttacctgacgcatcgaatagagaaaagattctgaaagtaatattggcaaaggaagagttggctccaggtattgatatggattctcttgccactatgaccgatggatattcaggaagtgacctgaaggtatgcttgataatgatgcgcttcccaaataacacaagtgattggaaatggttgcacaattttcactgacttgcttgtgttcccagaatctatgcgtgaccgcagctcattaccccattcgagaaatccttgagaaggaaaagaaggttggcattgcttgtaccttgctgtcaactgaagacctagataaagtttgttttttgggtttatgctgtctttagatgcactccttattcttctactataattatactgttaagtgacaacgctttttattttatatgctaatatggggttcgcttcctttctattcaggagaaaaatgtggcaaaagcagaaggcaggcctgagcctgcattgtatggtagtgaggacattcgtcctctgaccttagatgatttcaaatctgcccatgagcaggtgagaccagcacattttatatttattttgagctctttacttgaaagttcataatctgatcaatttatttaccacaggtttgtgcaagcgtttcatctgattcagcaaacatgaatgaacttcttcaatggaacgacctatatggcgagggcggttcgaggaagaagaaagcactgagctacttcatgtgagagcgattccatcataacatgtgagagagattaagaccctgccatattgatattgtacagtcgctataccacccctcttggcaagcgcttcgcgacttggccggccaaaaccagtcgggttttttcctcttctcccttcaggagcagcggccgccctaccttcgtcctccatcgtgtaattaaattgtgtactgaaaccagttcaggtttatttgtcagctgtttgttgctcgagttaacttgtaatgttatcattgccttaccccaatgccacctctttttttcccccctcttctgcatccttggctgtagtagcatatagtccttgtgtacctctgtacatgttgaaatcaaaggacatgggaataagaacattgtcctgttgtcgacagtataatgattctgactagccatgggttgttgacattataaggaggaactaagacgtctttgcgtcttaattttttttctatatatatcttgagctgatagattatct

SEQ ID NO: 10 - DNA, 2682 bp, Oryza sativa
atggtcgagcatatggattggcagccggtgaccaccctcggccccaatttctcgccggagctgcatagcctgctcctctccgaccaccgcgcgtccttgctctccctcctccgccgccaggacgacgagctccggaccaagatcaagaaccacctcctcgcgctcggatggaccatcgcctccaagcccaacccccctggccttgcgcccaggctccgctacgtctcccccgccggaaccaagtcctactactccctccgccgcctcatccagaccatccacctccaccaccatcctacccaatcccaatcccaatctcaatctgattcttgcggttgcggagatacccctcttcttcttgaggaatctgatgacgaccaataccaagaacaacaagaagatgatgccatcgcggggtacgttgccttcatggaggagcagaatgccaggcgcgaccgcgggcagggaaatgatgaggagcagaggtccatggccaaggagcttcggatcaaggctaaggatcagctcaggtcgtcgggatggacattctccatgaaggtcaagtacaacggccgcgaggagctgcgctacacagagcctcgtggccgctcccacatctccctcatcaccgcctgcaaggcctaccttcttcaacacacgccgtcgacgacaatggcttcatgcagcaacaacaacaacaagagaccagcgccacccgctgcctgcaagactgccacttctagcaagaagaacaagaagaagaaggcgagcctgcagcaggcgcgcgttctgcgtccacagccaagaaacgaggaggggaatgcgctgacgccggcgcgcgcgaggacgctactgtcattgctgatcgacaagaagatcctggcgccgagggaccagctcatctacaccacgaagcgagggctcatcaccggagacggcatggtgaaatgcatgtgcggcggctgcatcaacaacaacaacaagaggagggtagcagagtacacggtggccgagttcgcggtgcacggcgacggcgatgtcgcctcttcctcgtcacggcagccttgggcgcgcatgtttgtgggtgacgacaggtcactgtcgcagtgcctggtgcagctgatgatggcggacgacgaggcgggctccggcaggaagaagaagaagaagaagtatttgccgtatgtgtggcgtggtgcaagggtgaagcggaaatgggaggaggacgacgactacgtgtgctccgtgtgccacgactgcggcgagctgctcatgtgcgaccgctgtccgtccatgttccaccatgcctgcgtgggtttggagtccactccgcagggcgactggttctgcccggcctgcacctgcgccatctgcggctccagcgacttggacgatccaccagctaccaccaccacccaaggattcagcagcgacaggatggtcatctcctgcgagcagtgccgacgagagtgtaagtcactcgatcactcactaaatcatcattttgcatttttagttagctgctgatgattgatggagaattttcaaatggatgagttgatctcagatcatgttgggtgcatgcgagagagagacaatgggttgtggtatccagaggcggatgaagaggggccatggttgtgcagtgaggcctgctcaaagatatacctgctcctcgaggagctcgctgtcgtccaagccccttgtcgttcagttgcttcgggcttgtcattggtggtgttgagacgcggtgcagcgagagacggagaggaggaggagcacgccaagctgtgcatggccttggatgtgctccgcgagtgtttcgtcaccctcatcgagcctcgcacgcagaccgacctcactgccgacatcgtcttcaacacagagtacgcccgcaatatataattaagctctaataaagctccaactgtttaactgcaaaaagaaaaaaaagtcaattgattatcaatgggtcaattaattgttggctactctatatatggatgcagatcggagctgagacggctagatttccgggggttttacgtggtaggactggagaaagccggcgagcttatagccgtggctacactgaggtaagtaataatataatgtgtgtgtatccatctgcgttcctggctggcttccatgcatgatcaaatctcaactatagtcgatcaggtactaggaattaacaataagctgtttgtttgcgtcgtatatatgtgcagagtgtatggagaggaagttgcagaagtgccgctggtagggactagattcgcgcgtcgtcggcaagggatgtgccgccttctcatggatgagatacaaaaggtacgtactctctactagctagcttaatgatcaatcatcgagttattaatcgatctatcgttattaatcttacatgcatatattcgatgatgcatgcgtatgcagttgcttggtgaggtgggggtggagaggcttgtgctgccggcggtgccggagatggtggcgacatggacgggaccatcatttgggatcagagagatggggcaggctgaaaggcaggatgtagcacaccatgccatcctccgcttccagggaaccatcatgtgccacaagcagctacctcctcaacctcaacttgggcacacaacgacgaccccagcaggaagaattccaagtccaataccattgtaa

SEQ ID NO: 11 - DNA, 2920 bp, Oryza sativa
gcgcgctatggtcggcggcgagcccttgcggcggcggcggcatctcgccgacgatggattcttccggttcctcctcccctcgccgaagccggccaccaccaccaccaccacccctcctcccgccgcgctgttcgtcccgccccaccgcctcatcgcgccacccgtgccgctcccccagccaccccgccccgaggagcgcctcttcatcgtgccccccacgcgcccttcctggctcccgcctctctcgatacctccgccggcgacggcgacggccccgccgccgactcgttgccctccgcggcggatgggcaatggtggtggtggttgctttggcggccggagtggggtggtggggtggaggtatggaggtttcgtcgggaatggtggacatcgtggcttcgagcggcggagggtgggaggaggcttcatcggagcagcgaatgcaggcgaggccaccggcggcgagaggcgggcggtggtgcgtaagagggagaagaaggtgtgggtcgccgtggagaagaagggcgaggactgcggcggcggggatgaggaccaggcggcgatgggcgcgggctatgccggtggagatgagagggacgagcaggtggacgtcgacgacgacgaacaagacgacggcgacggcgatgaccctttcgacgtcgccgccgatcacgacctcctcgccgtcgtcgccgatggcgccggcagcgagaaaccaatggaggtagaggaaaactcgaccgagacttcaccgacgcttcatgctatgaaactcttcaccttgctctccctttcagcagctcggatcgccgccggatcaaccgccgccgccgccgccgcgccagcgagtcggaacgcggcggtggagggtggaacgccgccatgacatcgacgccttcacgcccggattgctctccctctacgaatccctcaatccatcagaggagcacaaggccaagcagaggcagctcatcgaatccctcacgaattctgtgagcaaagagtggcccaacgcccagctgcatctctatggatcgtgtgctaattccttcggaaattcgcatagcgatgtagatgtttgtctccaaattgacactgctgcagaagaaaacattgctgagcttcttctcgcattagcagagacattgcgcaaggatgatttcgacaatgttgaggtaattaattaattcttctccagatatatgcaattttgcaagcaagcatctcttccaaatttgaatgattcatcattacttgaaaattttaatattagctgcataattactctggaactggaatgtgaagagctagttgcctcatgtaattcttcataaatgccccggtggaaaaaaaattaggcccgggaaaaaataattcttcataattgatgtatttgaaaaatagcttcttaattgacgcaggttgagactgttcatcttctaatgcctgtcaaattcatgcaggctattaccagtgctcgagttccaattgttaagattgcagatccaggaagtggcttgtcatgtgatatttgtgtcaacaatctctttgctgttgctaacacaaagcttctcaaggattatgcccagatagatgagaagttgcttcagctggccttcattgtaaagcactgggccaaactaaggggcgttaatgaaacatatcgtggaaccctttcgagctatgcgtgagttaaacgactttgcgttcatgattttcccctttttacagctatattttatttgttgaaaatcttgaacctgatcttgttcttattaatttgcagatatgtgcttatgtgcattagcttcttgcagcaaagagagcccaagattctaccatgtttgcaggttctataccttactttttatctcaccaatatgtttcattcatatgaactactcgctgaagtactgatactttggatgcacacagtttaggataaaatcacttcacgcttgcttcatctgcatttagactcatgatgttgatacaggttaaacgaatggtctgcacacatttttattttcattttcatttttttatctctaaaatacttaacttctatctcttttatttgctggtggaatcgtcctgatttcgcattctcccttcatatatgcatgattctgttttggatggctcgacattcaactatggttggtcaattttctttcctttcttttcttttttttttcactaatgtagcaggtccattttttattctctttgattgatgttactaagagttgcttgcgtctagaattgttcagatgaaatcaggatctgcaaagttgtatatttcagcttcatctatgaattacttccgtcagttttatcctgatgcaatttgtcctctcaactcactccaattgtaggcgatggagccaacatatactctggttgtcgatggcactgaatgtgcatacttcgaccaagttgaccagcttaaagattttggtgctgaaaacaaggagagcatcgccgagttactgtgggcattcttccactactgggcattccatcacgattacaggaacgatgtcatctccgtccgcatggggaacacaatcaggtgagaattgccaacgagcgtttctcaaaaaccgactaaacttcaaaaattcgattttgaactaatgtcacatatgcttcaaagaatttcgcgtcctttttaacttggttcgatctctggtcgatcgatagcaagcaggagaagaactggacgactcgcgttggaaacgatcgccatctgatatgcatcgaggatcctttcgagaccagccatgacctcggtcgtgttgtcgacaggcagacaatcagggttctcagggaggagtttgagcgagctgccaccattctgcagtacgacgatgacccttgtgtggctctttttgagccctatgattatgaatcttga

SEQ ID NO: 12 - DNA, 4069 bp, Oryza sativa
gacgccgtccgtttcccttcttcttccgccgcgtctccccaacttccaagtcttccattcccccttcttccagtagcttcctcctccttctccgccgcaaccccacccaaccaaaccaaaaaaccaaaggaaaccaaagcaaagcgagctccgagctctccacccacgagcgcgtgcggaatctgtagtaaactagtaatccatccccgatcggcggcgatgcagaacccgccgagccaccccgttgacctgccgctggcggcggcgccgccgccggtgaaggcgccgaccccgcgcccccccacgccggcgtcgctgcagccggagtccccgggggtgttcttcacggccgccgcggcggccgccccggtcgggtcctcgcaccgccgcatcgccatcgccgttgacctctccgacgagtccgcgtacgccgtccgctgggccgtcgccaactacctccgccccggggacgccgttatactgctgcacgtccgccccacctccgtgctctacggcgccgactggggctccgtcgacctctccctccccgccgccaaccctaaccctagcggcgacccgccgtcggccgaggacgacgctgaggccgcggcccgcaagatggaggacgacttcgacgccttcaccgcgtccaaggccgacgacctggccaagccgctcaaggacgccgggatcccctacaagatccacatcgtcaaggatcacgacatgaaggagaggctctgcctcgaggtggagaggcttgggctcagcgcggtcatcatggggagcaagggcttcggcgcctccaggcggaccagcaaggggaggcttggaagcgtcagcgattactgcgtgcaccactgtgtgtgccccgtggtggtggtgcgtttccccgacgatggtgtggcggagggcggagaggccggtggcgcatcggagttggcggtgggcgaggaagtgctgcaccctgtgccagaggaggatgccgagtaccatgacgccactgaagagcacaagggtaattgatttaccgtctggtgtgcacttgtgttgctgactctatatgtgttgtttcctactttgatttgctatacaaagtgtaatctgaataatcaaatagtgggtatggataaaaaaacaaacgaacaatcaactacacctgcaaattatcgttattcgtgcaaaaataacgagaaaaatgagttgtaagataatgtaaggttgtgtagtacaacaatgaagcaatgttgacactcactgcagagccctatgttttggtttagttccccacaaccccaactccacaacatacataaaaccccttgacattttaggagtcgctctatgtagtcacagggaggtgcaataatatggtggggatgtggcttttaagtgtagggatatgctgtaacatactgcatttcctttctattctcaaaattacatgatctagtctattgttcatgacatctttatgtgcaaaagcgatttaactatacgattttgatcttgtcgtgtgatttagttttttggccaatcagttatcagtcaacaacagtgttatctatatatatcctatatagttggaaccaaaatctcaacatgcattgagccacatcatcctaaacatcctgaccgttggatcatctgttggagagctaaaacatattagctgatatgaaactaaagtatttaaatttagctatatttacatgcatttcatcgtcgtaattatggtacattcatgcagcttcaattttagttggaagactattgatctccccgccacataacaagagttaaagccctatacttgtttttttttcatcccttctttagcttgcatatcttttcaaagccgcttatccatacatagtcatcctgcagcaacgtgcgtggtaccatcttgtaaataaaggatatgcatggcacatacaagcctgtatacagggaacaggtggcgaatctgttaacaggtagttgcatgttatgtcgcataagtgcgcctgggtagtataatcacctcaaggtgggtgaccgcacaaatactactgttcatactgtgacgattagttaccgtttatatgttccttcagtgcaaagtactccctctatctcaaaatataaggcgcaactaccactaacgcaaagaccaagaaagaattattatcacctccttgtttggatcactcaaataaatacaatgcatgcacccaacgggattagagtttttgagggtggaggatataaaaaacgaattaaagaggagggaggtgctagttaattgtatgcatgcatgcacgccttatattacgaaacattgaaaaaaaagtggttatgcctaatatttttggatgggggagtatatttttacaccacgtgcatatctattaggcatgttcctagatagcttttggcacaagttgaacatgtctacaggttcttgttggttttcaaaactatggattggcataggaaataattacgatttggattcttttggatgttttgttatctgcatgcttcaaaatggataacataagtctatgattaattttaggtattggttgcatatgagtttataagggactgaaaactgtagttggagtttttcgccttgaactttacttatgagcaatgttcatgatggattgaattatgccattcatttgctgtatcatccattcgtgccaaaggttctgatttactaatcatgctaatgtcttaagttggttatgtttctgcagatacttgatcatgctgtatgatgatgtcctctttgatccaggtgagcatatcatgaattcttctactaagtttgtatggtcagtcttatggtttcaactgtgaaatgtttgttcggagaaattcatatatccaaaggatctaaagttctattttgtgctgtacatttctgtatatgtagtacacaggttaatctgttaagaccaaagaccataatatctctgtagtttgttgtaatgtgactagggctatataccaaattacaatttaaatggattaatcagattaacaactgaagcctcctttcacgtatttatcatgatgacaaaggctgtggtgttgggcaaaatgattattccattctccaaggctacacaggagaaacccagatgggcctgaagcctggacactgttatatttagagcccagtggttcactgttatatttggagctcagtggtcaagcctttgcactcttgataaaccttgatttcgttagatccgattgataattttccttccatctgactagctctctgacatattctaaattattcccacaatgccattatctgttccatatatggtaaattagaatatttgggaactgcaatgccctaacactgatttgagtgttttacaatacacttgttgaattaatttccttccgcttgtattcagtacctaatttcttccctttccttctgcagcatgtaaaggttctagcagtcaactgtttgctgggtgctaggtggggtttcttctgagggcatttgctgttccgttcaactttcactgcctatgtttattcgtgctttcactttgttgtattttattgtcttgtttcatgtagttttatctgacctactgcttgcacggttaatttttatgatttagaatttttgtgctttctcaacatcacaagatctgtttctgatgaaagttacagtttgactacttaacgaaccaagtaaccgtctcccttttatttttctcccttgttgtactttcccttaaagtaagattatggtagatatcatgaatctcttgatgtgcgcagtcaatatctgccaaaggaaactttttttttttaatctactgttaggaaaagttggcgtttctacataagagcatggttacttaaattgtgttctgctctctggtccctgtcttggtttgtttgttctgctgttagaattatagttaactcgtaactaggacttggaagctgagctctggaatcttctttgtacatgtataatctattggcacatggaaagtagtatgaccggtactcatctaatatgccttgctataaaattaag

SEQ ID NO: 13 - DNA, 2214 bp, Oryza sativa
actctcgggattcactcccgctgtccgtcctcgtttccgccccccacacaccggcccagcaacgcggcccgaggcttcctctcctcgcctcgcgaccgctccgccaaacctccgccgccgccggcgacgacgggcggccaaccgccccaccgtcgagtatggcctcatccccgtcaccggtctcgcccccctcggcgccctccacacaggtccgcctctcgccctttcgcctcctcgatcgtacgtcgccatctacgtttcctcgtactggtatttccccccgcagcttctttggtctgttcctccgaatccccgatccggtttggtgtatctggtggtggccatagcatgctcggttgttggtttcatctggttgtttggcttgagaaattgtgccaaatggtggcgcctgcatgttgcgactatatgcgtggatgcggtttcagagttcagaataggggtttggttattccactaggttattccgtaggatgaaatctttaatcaaatagtatcgtcaatgccaagaacaggtgagggaaggatcgatttagagccttaacttaatctgatgaactgaaattgtgccattttggatagaaattgtaggcgtgttgctttgtactgctgtgaatacatgaagcctgaagtttgtttgtcagcaacgggagtttatgggctgtaaagaaataaaaaagatcagcttgcattggtttctcaaaattttgtttgagatccaagaggaatttagaagcatgaaggataatggaatccgtccaataacaaaaaatgcatatactttagattgagaacataagagttaatgtattacactatttaatacttttgcagttcttaagttctgatgacattttatacattagcgaaggggggctgcagggaatggagactgtagagggttataagagaaagaggaaaatgaggacttttctacttagagctgtctgaaccatttcttcacatcactgggtctatgaaacaattttagatttcttcacaaaatgctgcgcaatttttttaaatttctgccccttctaaaaactgaatgcagaatctttttctatggttgcttttgtgcagccaaacataagtgtgccttatactacatggcatctgcattataccaaaagtggccttaccatatggactctatgcaaaatataacttccaacttctaagaagcagcttttatggactgtaaagaaaaaaacaatgtagcggtctttattatttctatttgtgcgttgggaagttgttctgttatcctatccactagaagtatgcatcatgtcattaacatccattttccattagtttgtgcctgatagaattaaaatttacattccaaatgcatattttaggatcagaaaaattattttgcttgatatacaatagtaatgaatggtaacgttcttatttcaatagagaaaacgaggctcctccacagactccattggcatgtatgcagttcaatgttgtgagtgtcacaaatggcgcaaggttccgacgaaggatgaatttgagacaattcgtgagaatttcactgaggagccatggcactgcagtagaagacctgactgctcgtgtgaagaccctgctgacatcgaatatgatagcagccgtatatgggtccttgacaagcctaacataccaaagcctccagcaggaactgagagactagtgattatgagaggtgatttgtctaaaatggatacctactatgtcatgccaaatgggaagcgtgtaaggtgcactgcagaggtggataagttccttgaggccaacccgcagtacaaagatcgcttttcagttgaaagcttcagctttacaacaccaaagattgtcgaggaaactgtttctcacaactccgtgtggaagtctggaaaggctaagaagcaggacaagataaatgctttgagcaataacaattaatttctttcaaaagactttatattatgcttaggtgatgctcatctcaggttgtggtgctaccctcatctactttgtgggctggtcaaatctgtttatatttgcacttagtctgttcggtctctgtgatctgtgtgttatgatgcgctgaaccttttctttcttcttcattatgtattaactcttggcaaactttgagatggttgtgaagggttgtgggtatgttggccataactaaccttagttataggcatgctcttttgt

SEQ ID NO: 14 - protein, 246 aa, Oryza sativa
MDNQQLPYAGQPAAAGAGAPVPGVPGAGGPPAVPHHHLLQQQQAQLQAFWAYQRQEAERASASDFKNHQLPLARIKKIMKADEDVRMISAEAPVLFAKACELFILELTIRSWLHAEENKRRTLQRNDVAAAIARTDVFDFLVDIVPREEAKEEPGSALGFAAGGPAGAVGAAGPAAGLPYYYPPMGQPAPMMPAWHVPAWDPAWQQGAAPDVDQGAAGSFSEEGQQGFAGHGGAAAGFPPAPPSSE

SEQ ID NO: 15 - protein, 742 aa, Oryza sativa
MMGTAHHNQTAGSALGVGVGDANDAVPGAGGGGYSDPDGGPTSGVQPPPQVCWERFIQKKTIKVLLVESDDSTRQVVSALLRHCMYEVIPAENGQQAWTYLEDMQNSIDLVLTEVVMPGVSGISLLSRIMNHNICKNIPVIMMSSNDAMGTVFKCLSKGAVDFLVKPIRKNELKNLWQHVWRRCHSSSGSGSESGIQTQKCAKSKSGDESNNNSGSNDDDDDDGVIMGLNARDGSDNGSGTQAQSSWTKRAVEIDSPQAMSPDQLADPPDSTCAQVIHLKSDICSNRWLPCTSNKNSKKQKETNDDFKGKDLEIGSPRNLNTAYQSSPNERSIKPTDRRNEYPLQNNSKEAAMENLEESSVRAADLIGSMAKNMDAQQAARATNAPNCSSKVPEGKDKNRDNIMPSLELSLKRSRSTGDGANAIQEEQRNVLRRSDLSAFTRYHTPVASNQGGTGFVGSCSPHDNSSEAMKTDSAYNMKSNSDAAPIKQGSNGSSNNNDMGSTTKNVVTKPSTNKERVMSPSAVKANGHTSAFHPAQHWTSPANTTGKEKTDEVANNAAKRAQPGEVQSNLVQHPRPILHYVNFDVSRENGGSGAPQCGSSNVFDPPVEGHAANYGVNGSNSGSNNGSNGQNGSTTAVNAERPNMEIANGTINKSGPGGGNGSGSGSGNDMYLKRFTQREHRVAAVIKFRQKRKERNFGKKVRYQSRKRLAEQRPRVRGQFVRQAVQDQQQQGGGREAAADR

SEQ ID NO: 16 - protein, 287 aa, Oryza sativa
MGMANEESPNYQVKKGGRIPPPRSSLIYPFMSMGPAAGEGCGLCGADGGGCCSRHRHDDDGFPFVFPPSACQGIGAPAPPVHEFQFFGNDGGGDDGESVAWLFDDYPPPSPVAAAAGMHHRQPPYDGVVAPPSLFRRNTGAGGLTFDVSLGGRPDLDAGLGLGGGSGRHAEAAASATIMSYCGSTFTDAASSMPKEMVAAMADVGESLNPNTVVGAMVEREAKLMRYKEKRKKRCYEKQIRYASRKAYAEMRPRVRGRFAKEADQEAVAPPSTYVDPSRLELGQWFR

SEQ ID NO: 17 - protein, 484 aa, Oryza sativa
MNCGPPDQLPPATAPSCFLNLNWDQSMDAAAGGHLDPALSSMVSSPASNSTGALHGISPQPHYGGGTPLSSPPKLNLSMMGQFHHYAAPPQVGGGGGGGGGLPILENLMPMGHLDQFLADPGFAERAARLSGFDARGGGGGGGYGGAGPAQFGLPDAGAAGASKEMELGNTRDESSVSDPAPGGAEIPPKGASDGNARKRKASGKGKGKDSPMSTSAAKEDSSGKRCKSTEESNAAAEENSGKGKAAQSNSENGGGKKQGKDSSSKPPEPPKDYIHVRARRGEATDSHSLAERVRREKISQRMKLLQDLVPGCNKVVGKAVMLDEIINYVQSLQRQVEFLSMKLATVNPQLDFNNLPNLLAKDMHQSCSPLQSSHFPLETSGAPLPYINQPQQGNPLGCGLTNGMDNQGSMHPLDPAFCRPMGSHHPFLNGVSDAASQVGAFWQDDLQSVVQMDMGQSQEIATSSNSYNGRIVANSPHENGALT

SEQ ID NO: 18 - protein, 172 aa, Oryza sativa
MTELFDTAVTSLLHLPEVLDRLGAAAGDRRSAGDHAHHAAHGHGQHRISGIGSGAPVDIMETPGEYAFVLDVPGLSKSDIQVTLEEDRVLVMKSSNGAGNGKRKREEEEGECKYIRLERRASPRAFARKFRLPEDADTGGISARCENGVLTVTVKKRPPPEKKTKSVQVTIA

SEQ ID NO: 19 - protein, 450 aa, Oryza sativa
MREIISIHIGQAGIQVGNACWELYCLEHGIEPDGTMPSDTTVGVAHDAFNTFFSETGAGKHVPRAIFVDLEPTVIDEVRTGSYRQLFHPEQLISGKEDAANNFARGHYTVGKEIVDLCLDRVRKLADNCTGLQGFLVFNAVGGGTGSGLGSLLLERLSVDYGKKSKLGFTIYPSPQVSTAVVEPYNSVLSTHSLLEHTDVAVLLDNEAIYDICRRSLDIERPTYTNLNRLISQIISSLTTSLRFDGAINVDVTEFQTNLVPYPRIHFMLSSYAPVISAEKAYHEQLSVPEITNAVFEPSSMMAKCDPRHGKYMACCLMYRGDVVPKDVNAAVATIKTKRTVQFVDWCPTGFKCGINYQPPSVVPGGDLAKVQRAVCMISNNTAVAEVFSRIDHKFDLMYAKRAFVHWYVGEGMEEGEFSEAREDLAALEKDYEEVGAEGADDENDDGEDY

SEQ ID NO: 20 - protein, 316 aa, Oryza sativa
MGMGMRRERDAEAELNLPPGFRFHPTDDELVEHYLCRKAAGQRLPVPIIAEVDLYKFDPWDLPERALFGAREWYFFTPRDRKYPNGSRPNRAAGNGYWKATGADKPVAPRGRTLGIKKALVFYAGKAPRGVKTDWIMHEYRLADAGRAAAGARKGSLRLDDWVLCRLYNKKNEWEKMQQGKEVKEEASDMVTSQSHSHTHSWGETRTPESEIVDNDPFPELDSFPAFQPAPPPATAMMVPKKESMDDATAAAAAATTIPRNNSSLFVDLSYDDIQGMYSGLDMLPPGDDFYSSLFASPRVKGTTPRAGAGMGMVPF

SEQ ID NO: 21 - protein, 206 aa, Oryza sativa
MASRDAATFQVYRPMAMPTPAALPPSSQQITMPFTAAPVDAVLPAPRKAAATQGGKDRHSKVNGRGRRVRMPIVCAARVFQLTRELGLKSDGQTIEWLLRQAEPSILAATGSGTTPAVFSCSSAPSTASSSFLLGKRPRQEDHEAPTFWEALQQQPRPAVSSWGALVSPSQEAQAYASSVAQVHHLNLLSALSGAATRRPAQEESR

SEQ ID NO: 22 - protein, 1202 aa, Oryza sativa
MVETRRSSAAAASKRSSPSPSSSSAPPPKRPKAEAAPASPTASVPGRIEEDSAATKSAGSGEDAAAKRDQGGDKAAVAVVESSRKKKEQQQQQQQQQQQQATPWAKLLSQSSQSPHLPISVPQFSVGQNKSCNLWLKDQPVSKILCRLRQLEQGTCELEVLGKKGTVQLNGRSITAGTKVPLKGGDEVVFSPCGKHAYIFQHPLNDKIPKMVPPSPVTLLEPPVAGVKRLRMENRTGDTSAVAGTELLASVSDQLKDLSAASPASAGENNQRLVRPMASSASDKSKGNGIIPDKECENGENANEVNSNVEDSPLDVAAAPVVSPDAVPNDISQHNGFGSDAHLGAEIALEDQRDLIRHLNSSASLPPSRCQAFKDGMKQGIISPNDIDVTFENFPYYLSDNTKNVLLSCAFIHLEKKEFIKQFSEISSINQRILLSGPAGSEIYQETLIKALAKHFGARLLVVDSLLLPGAPSKDPESQKDAAKSDKSGDKAGSEKLAILHKNRSSLADAMHFRRPAVQPSSVHADIVGTSTLHSASLPKQESSTATSKSYTFREGDRVRYVGPAQQSSLSQRGPSYGYRGRVMLAFEENGSSKIGVRFDKQIPDGNDLGGLCEEDHGFFCSADLLRPDFSGGEEVERLAMAELIEVISEEHKAGPMIVLLKDVEKSFTGITESLSSLRNKLEALPSGVLIIGSHTQMDSRKEKAHPGGFLFTKFASSSQTLFDLFPDSFGSRLHERNKESPKAMKHLNKLFPNKISIQLPQDETLLTDWKQQLDRDVETLKAKSNVGSIRTFLSRNGIECSDLEELFIKDQSLTNENVDKIVGYAVSYHLKHNKVEISKDGKLVLASESLKHGLNMLQNMQSDNKSSKKSLKDVVTENEFEKRLLADVIPPNDIGVTFDDIGALENVKDTLKELVMLPLQRPELFCKGQLTKPCKGILLFGPPGTGKTMLAKAVATEAGANFINISMSSITSKWFGEGEKYVKAVFSLASKIAPSVIFIDEVDSMLGRRENPGEHEAMRKMKNEFMVNWDGLRTKDKERVLVLGATNRPFDLDEAVIRRFPRRLMVNLPDASNREKILKVILAKEELAPGIDMDSLATMTDGYSGSDLKNLCVTAAHYPIREILEKEKKEKNVAKAEGRPEPALYGSEDIRPLTLDDFKSAHEQVCASVSSDSANMNELLQWNDLYGEGGSRKKKALSYFM

SEQ ID NO: 23 - protein, 744 aa, Oryza sativa
MVEHMDWQPVTTLGPNFSPELHSLLLSDHRASLLSLLRRQDDELRTKIKNHLLALGWTIASKPNPPGLAPRLRYVSPAGTKSYYSLRRLIQTIHLHHHPTQSQSQSQSDSCGCGDTPLLLEESDDDQYQEQQEDDAIAGYVAFMEEQNARRDRGQGNDEEQRSMAKELRIKAKDQLRSSGWTFSMKVKYNGREELRYTEPRGRSHISLITACKAYLLQHTPSTTMASCSNNNNKRPAPPAACKTATSSKKNKKKKASLQQARVLRPQPRNEEGNALTPARARTLLSLLIDKKILAPRDQLIYTTKRGLITGDGMVKCMCGGCINNNNKRRVAEYTVAEFAVHGDGDVASSSSRQPWARMFVGDDRSLSQCLVQLMMADDEAGSGRKKKKKKYLPYVWRGARVKRKWEEDDDYVCSVCHDCGELLMCDRCPSMFHHACVGLESTPQGDWFCPACTCAICGSSDLDDPPATTTTQGFSSDRMVISCEQCRREYHVGCMRERDNGLWYPEADEEGPWLCSEACSKIYLLLEELAVVQAPCRSVASGLSLVVLRRGAARDGEEEEHAKLCMALDVLRECFVTLIEPRTQTDLTADIVFNTESELRRLDFRGFYVVGLEKAGELIAVATLRVYGEEVAEVPLVGTRFARRRQGMCRLLMDEIQKLLGEVGVERLVLPAVPEMVATWTGPSFGIREMGQAERQDVAHHAILRFQGTIMCHKQLPPQPQPQPQLGHTTTTPAGRIPSPIPL

SEQ ID NO: 24 - protein, 597 aa, Oryza sativa
MVGGEPLRRRRHLADDGFFRFLLPSPKPATTTTTTPPPAALFVPPHRLIAPPVPLPQPPRPEERLFIVPPTRPSWLPPLSIPPPATATAPPPTRCPPRRMGNGGGGCFGGRSGVVGWRYGGFVGNGGRRGFERRRVGGGFIGAANAGEATGGERRAVVRKREKKVWVAVEKKGEDCGGGDEDQAAMGAGYAGGDERDEQVDVDDDEQDDGDGDDPFDVAADHDLLAVVADGAGSEKPMEQLGSPPDQPPPPPPRQRVGTRRWRVERRHDIDAFTPGLLSLYESLNPSEEHKAKQRQLIESLTNSVSKEWPNAQLHLYGSCANSFGNSHSDVDVCLQIDTAAEENIAELLLALAETLRKDDFDNVEAITSARVPIVKIADPGSGLSCDICVNNLFAVANTKLLKDYAQIDEKLLQLAFIVKHWAKLRGVNETYRGTLSSYAYVLMCISFLQQREPKILPCLQAMEPTYTLVVDGTECAYFDQVDQLKDFGAENKESIAELLWAFFHYWAFHHDYRNDVISVRMGNTISKQEKNWTTRVGNDRHLICIEDPFETSHDLGRVVDRQTIRVLREEFERAATILQYDDDPCVALFEPYDYES

SEQ ID NO: 25 - protein, 275 aa, Oryza sativa
MQNPPSHPVDLPLAAAPPPVKAPTPRPPTPASLQPESPGVFFTAAAAAAPVGSSHRRIAIAVDLSDESAYAVRWAVANYLRPGDAVILLHVRPTSVLYGADWGSVDLSLPAANPNPSGDPPSAEDDAEAAARKMEDDFDAFTASKADDLAKPLKDAGIPYKIHIVKDHDMKERLCLEVERLGLSAVIMGSKGFGASRRTSKGRLGSVSDYCVHHCVCPVVVVRFPDDGVAEGGEAGGASELAVGEEVLHPVPEEDAEYHDATEEHKATCVVPSCK

SEQ ID NO: 26 - protein, 185 aa, Oryza sativa
MASSPSPVSPPSAPSTQRKRGSSTDSIGMYAVQCCECHKWRKVPTKDEFETIRENFTEEPWHCSRRPDCSCEDPADIEYDSSRIWVLDKPNIPKPPAGTERLVIMRGDLSKMDTYYVMPNGKRVRCTAEVDKFLEANPQYKDRFSVESFSFTTPKIVEETVSHNSVWKSGKAKKQDKINALSNNN

SEQ ID NO: 27 - DNA, 24 bp, Oryza sativa
agcagcgccggatgtggaccaggg

SEQ ID NO: 28 - DNA, 23 bp, Oryza sativa
aaccagcagctaccctacgccgg

SEQ ID NO: 29 - DNA, 24 bp, Oryza sativa
aacgacgccgtgcctggggctggg

SEQ ID NO: 30 - DNA, 24 bp, Oryza sativa
ccggatggcggaccaacctccggt

SEQ ID NO: 31 - DNA, 24 bp, Oryza sativa
atgaggagtcgccaaattatcagg

SEQ ID NO: 32 - DNA, 24 bp, Oryza sativa
ccggcgggctcacgttcgacgtct

SEQ ID NO: 33 - DNA, 23 bp, Oryza sativa
ccaagggggcttccgacggcaat

SEQ ID NO: 34 - DNA, 20 bp, Oryza sativa
aggagaattccggcaagggg

SEQ ID NO: 35 - DNA, 24 bp, Oryza sativa
agcccaagttggtagccatgacgg

SEQ ID NO: 36 - DNA, 23 bp, Oryza sativa
ccaagtccgacatccaggtgcgt

SEQ ID NO: 37 - DNA, 22 bp, Oryza sativa
atcggccaggccgggatccagg

SEQ ID NO: 38 - DNA, 23 bp, Oryza sativa
accctgagcagctcatctctggg

SEQ ID NO: 39 - DNA, 23 bp, Oryza sativa
atctctacaagttcgacccgtgg

SEQ ID NO: 40 - DNA, 22 bp, Oryza sativa
actcgcacacccactcgtgggg

SEQ ID NO: 41 - DNA, 25 bp, Oryza sativa
accttccaggtctaccggcccatgg

SEQ ID NO: 42 - DNA, 20 bp, Oryza sativa
agctcacccgcgagctgggg

SEQ ID NO: 43 - DNA, 24 bp, Oryza sativa
cctatcagtgttcctcagttttct

SEQ ID NO: 44 - DNA, 21 bp, Oryza sativa
ccagagtcgcagaaagatgct

SEQ ID NO: 45 - DNA, 24 bp, Oryza sativa
ccttgcgcccaggctccgctacgt

SEQ ID NO: 46 - DNA, 25 bp, Oryza sativa
agctcatctacaccacgaagcgagg

SEQ ID NO: 47 - DNA, 26 bp, Oryza sativa
atggtggacatcgtggcttcgagcgg

SEQ ID NO: 48 - DNA, 23 bp, Oryza sativa
ccttcggaaattcgcatagcgat

SEQ ID NO: 49 - DNA, 22 bp, Oryza sativa
ccggagtccccgggggtgttct

SEQ ID NO: 50 - DNA, 24 bp, Oryza sativa
accactgtgtgtgccccgtggtgg

SEQ ID NO: 51 - DNA, 24 bp, Oryza sativa
cctgactgctcgtgtgaagaccct

SEQ ID NO: 52 - DNA, 24 bp, Oryza sativa
actgtttctcacaactccgtgtgg

SEQ ID NO: 53 - DNA, 24 bp, Oryza sativa
attcacacgttacaaacgcacggg

SEQ ID NO: 54 - DNA, 26 bp, Oryza sativa
acctttgtggtgcttccttgtctagg

SEQ ID NO: 55 - DNA, 25 bp, Oryza sativa
atcggttacgaatcaatcctctcgg

SEQ ID NO: 56 - DNA, 23 bp, Oryza sativa
gtgcgctctgttcctggattcgg

SEQ ID NO: 57 - DNA, 24 bp, Oryza sativa
agaagaaggcggattcctttttgg

SEQ ID NO: 58 - DNA, 25 bp, Oryza sativa
aagaggccgggaaacctagaccagg

SEQ ID NO: 59 - DNA, 25 bp, Oryza sativa
actgccccacatggtccgcatttgg

SEQ ID NO: 60 - DNA, 25 bp, Oryza sativa
tgtttctcatgcatgaaggataagg

SEQ ID NO: 61 - DNA, 22 bp, Oryza sativa
ggcccagcaacgcggcccgagg

SEQ ID NO: 62 - DNA, 24 bp, Oryza sativa
atggttttgaatcgattgacctgg